(19) United States
(12) Patent Application Publication
(10) Pub. No.: US 2021/0137078 A1
(43) Pub. Date: May 13, 2021

(54) LIVESTOCK SENSOR DEVICE, LIVESTOCK ASTASIA INFERENCE METHOD, LIVESTOCK ASTASIA INFERENCE PROGRAM, AND LIVESTOCK MANAGEMENT SYSTEM

(71) Applicant: SONY CORPORATION, TOKYO (JP)

(72) Inventors: HIDETATSU YAMAMOTO, KANAGAWA (JP); SEIJI OHISHI, KANAGAWA (JP); HIDETOSHI ISAWA, KANAGAWA (JP); KAZUHIKO TSUJI, KANAGAWA (JP); NAOKI TAMAI, KANAGAWA (JP); HIDEAKI KAMEI, KANAGAWA (JP); YOSHINORI USAMI, KANAGAWA (JP); TAKESHI NEGORO, KANAGAWA (JP); KOICHI YAMAGUCHI, KANAGAWA (JP); KAZUYA TERASAKI, KANAGAWA (JP); OSAMU MIKI, KANAGAWA (JP); KENJI SASAKI, CHIBA (JP); TOMOYA IMAMURA, KANAGAWA (JP); MAMI MAMIYA, KANAGAWA (JP)

(21) Appl. No.: 16/623,871

(22) PCT Filed: Jul. 10, 2018
(86) PCT No.: PCT/JP2018/026028

§ 371 Date: Dec. 18, 2019

(30) Foreign Application Priority Data
Aug. 22, 2017 (JP) 2017-159020

(51) Int. Cl. A01K 29/00; A01K 11/00; G06Q 50/02

(52) U.S. Cl. CPC A01K 29/005; G06Q 50/02; A01K 11/006

(57) ABSTRACT

Provided are a livestock sensor device, a livestock astasia inference method, a livestock astasia inference program, and a livestock management system that are capable of preventing damage to stockbreeders. According to an embodiment of the present technology, there is provided a livestock sensor device including a postural-state determination unit, a state inference unit, a transmission unit, and a casing. The postural-state determination unit determines in which of a prostrate state and a non-prostrate state a livestock animal has been on a basis of output values from an acceleration sensor. The state inference unit infers whether or not the livestock animal has developed astasia on a basis of a duration of the prostrate state. The transmission unit transmits, to a server, an astasia-notification data item indicating that an inference that the livestock animal has developed the astasia is made when the inference that the livestock animal has developed the astasia is made. The casing is configured to be capable of housing the acceleration sensor, the postural-state determination unit, the state inference unit, and the transmission unit, and capable of being attached to a head of the livestock animal.

### TECHNICAL FIELD

The present technology relates to a livestock sensor device, a livestock astasia inference method, a livestock astasia inference program, and a livestock management system.

### BACKGROUND ART

There have been made attempts to properly manage livestock animals by introducing devices or systems for managing the livestock animals into stockbreeding facilities.

For example, Patent Literature 1 discloses a livestock/poultry feeding management system that enables prediction of growth and the meat quality in the shipping stage by analyses of growth data items of the livestock animals and environment data items.

In addition, Patent Literature 2 discloses a livestock management system in which sensor devices capable of energy harvesting are attached to the livestock animals, and inferences in what state the livestock animals have been are made on the basis of information items about the power generation.

### CITATION LIST

### Patent Literature

Patent Literature 1: Japanese Patent Application Laid-open No. H7-8128

Patent Literature 2: International Publication No. WO 2016/181604 A1

### DISCLOSURE OF INVENTION

### Technical Problem

Meanwhile, with regard to the livestock animals such as beef cattle, their health conditions may be rapidly degraded in a case where astasia continues, which may cause serious damage to stockbreeders such as livestock farmers.

However, devices and the like, which are capable of accurately inferring whether or not the livestock animals have developed the astasia, have never been known.

In view of such circumstances, the present technology has been made to achieve an object to provide a livestock sensor device, a livestock astasia inference method, a livestock astasia inference program, and a livestock management system that are capable of preventing damage to stockbreeders.

### Solution to Problem

In order to achieve the above-mentioned object, according to an embodiment of the present technology, there is provided a livestock sensor device including a postural-state determination unit, a state inference unit, a transmission unit, and a casing.

The postural-state determination unit determines in which of a prostrate state and a non-prostrate state a livestock animal has been on a basis of output values from an acceleration sensor.

The state inference unit infers whether or not the livestock animal has developed astasia on a basis of a duration of the prostrate state.

The transmission unit transmits, to a server, an astasia-notification data item indicating that an inference that the livestock animal has developed the astasia is made when the inference that the livestock animal has developed the astasia is made.

The casing is configured to be
capable of housing
   the acceleration sensor,
   the postural-state determination unit,
   the state inference unit, and
   the transmission unit, and
capable of being attached to a head of the livestock animal.

According to another embodiment of the present technology, there is provided a livestock astasia inference method including the steps of:

determining in which of a prostrate state and a non-prostrate state a livestock animal has been on a basis of output values from an acceleration sensor; and

inferring whether or not the livestock animal has developed astasia on a basis of a duration of the prostrate state.

According to yet another embodiment of the present technology, there is provided a program for causing a computer to carry out a livestock astasia inference method including:

determining in which of a prostrate state and a non-prostrate state a livestock animal has been on a basis of output values from an acceleration sensor; and

inferring whether or not the livestock animal has developed astasia on a basis of a duration of the prostrate state.

According to yet another embodiment of the present technology, there is provided a livestock management system including a postural-state determination unit, a state inference unit, a notification-information generation unit, and a notification unit.

The postural-state determination unit determines in which of a prostrate posture and a non-prostrate posture a livestock animal has been on a basis of output values from an acceleration sensor.

The state inference unit infers whether or not the livestock animal has developed astasia on a basis of a duration of the prostrate posture.

The notification-information generation unit generates an astasia-notification information item including an astasia-notification data item indicating that an inference that the livestock animal has developed the astasia is made when the inference that the livestock animal has developed the astasia is made.

The notification unit notifies a user of the astasia-notification information item. Advantageous Effects of Invention

As described above, according to the present technology, it is possible to provide the livestock sensor device, the livestock astasia inference method, the livestock astasia inference program, and the livestock management system that are capable of preventing damage to stockbreeders.

### BRIEF DESCRIPTION OF DRAWINGS

FIG. 1 A schematic view illustrating a schematic configuration of a livestock management system according to a first embodiment of the present technology.

FIG. 2 A view illustrating an external appearance of a sensor device included in the livestock management system.

FIG. 3 A view illustrating how the sensor device is attached to a cattle individual as a livestock animal.

FIG. 4 A block diagram showing respective hardware configurations of devices included in the livestock management system.

FIG. 5 A view illustrating an example in which the sensor device is attached to the livestock animal.

FIG. 6 A block diagram showing a functional configuration of the livestock management system.

FIG. 7 An explanatory view illustrating postural states of the livestock animal and values of accelerations along detection axes.

FIG. 8 A chart showing results of an experiment for investigating a duration of a prostrate state.

FIG. 9 A flowchart showing an operation example of a detection-signal output procedure by the sensor device.

FIG. 10 A flowchart showing an operation example of an astasia inference procedure by the sensor device.

FIG. 11 A flowchart showing an operation example of an astasia notification procedure in the livestock management system.

FIG. 12 An exemplary view showing a screen of a livestock management application, which is displayed on a user terminal included in the livestock management system.

FIG. 13 An exemplary view showing other screens of the livestock management application, which are displayed on the user terminal included in the livestock management system.

FIG. 14 An exemplary view showing still other screens of the livestock management application, which are displayed on the user terminal included in the livestock management system.

FIG. 15 A view illustrating how a sensor device according to Modification 1 of the embodiment is attached to the cattle individual as the livestock animal.

FIG. 16 A view illustrating how a sensor device according to Modification 2 of the embodiment is attached to the cattle individual as the livestock animal.

FIG. 17 A schematic view illustrating a schematic configuration of a livestock management system according to a second embodiment of the present technology.

FIG. 18 A block diagram showing a functional configuration of the livestock management system.

FIG. 19 A flowchart showing an operation example of a postural-data accumulation procedure in the livestock management system.

FIG. 20 A block diagram showing a functional configuration of a livestock management system according to a third embodiment of the present technology.

FIG. 21 A block diagram showing respective hardware configurations of devices included in a livestock management system according to a fourth embodiment of the present technology.

FIG. 22 A block diagram showing a functional configuration of the livestock management system.

FIG. 23 A flowchart showing an operation example of a vibration-detection-signal output procedure by a sensor device included in the livestock management system.

FIG. 24 A flowchart showing an operation example of a self-standing-attempt-state inference procedure in which a vibration detection signal from the sensor device is used.

FIG. 25 A block diagram showing a functional configuration of a livestock management system according to another embodiment of the present technology.

FIG. 26 A block diagram showing a functional configuration of a livestock management system according to still another embodiment of the present technology.

FIG. 27 A block diagram showing a functional configuration of a livestock management system according to yet another embodiment of the present technology.

FIG. 28 A block diagram showing a functional configuration of a livestock management system according to yet another embodiment of the present technology.

### MODE(S) FOR CARRYING OUT THE INVENTION

Now, with reference to the drawings, embodiments of the present technology are described.

### First Embodiment

[Summary of Livestock Management System]

FIG. 1 is a schematic view illustrating a schematic configuration of a livestock management system according to a first embodiment of the present technology.

A livestock management system 100 according to this embodiment is configured to be capable of executing an astasia inference procedure of inferring whether or not a livestock animal has developed astasia, and executing an astasia notification procedure of notifying a user that an inference that the astasia has developed is made.

The livestock management system 100, which is introduced, for example, to stockbreeding facilities, can be utilized by stockbreeders (users). The stockbreeding facilities are not particularly limited as long as the livestock animals can be accommodated therein. Stockbreeding facilities for beef cattle typically include a barn (cattle shed) including a plurality of stalls each capable of accommodating several cattle.

As examples of the livestock animals, there may be mentioned industrial animals such as the beef cattle, cows, pigs, horses, sheep, goats, and poultry, and pets such as dogs, cats, and rabbits. In this embodiment, the beef cattle are taken as an example. Hereinbelow, the beef cattle are also simply referred to as “cattle.”

Of the beef cattle, fed cattle being fed are particularly liable to develop the astasia if remaining in a prostrate posture. In addition, by the astasia, these livestock animals are liable to develop diseases such as bloat, and even may end up dead in some cases. The present technology enables inference and notification that the livestock animal has developed the astasia to the users. With this, damage to be caused by the astasia of the livestock animals can be prevented.

As illustrated in FIG. 1, the livestock management system 100 includes a sensor device 1, a relay device 2, a server 3, and a user terminal 4.

The sensor device 1 is attached to a livestock animal A. The sensor device 1 is capable of executing the astasia inference procedure of inferring whether or not the livestock animal A has developed the astasia, and transmitting an astasia-notification data item described later.

The relay device 2 receives the astasia-notification data item from the sensor device 1, and transmits this astasia-notification data item to the server 3 via a network N.

The server 3 generates an astasia-notification information item including the astasia-notification data item, and transmits this astasia-notification data item to the user terminal 4 via the network N.

The user terminal 4 notifies the user of the received astasia-notification information item.

This enables the user to grasp, via the user terminal 4, that the livestock animal A is likely to have developed the astasia, and the user to provide prompt treatment.

The livestock management system 100 may include a plurality of sensor devices 1 (refer to FIG. 17). All the sensor devices 1 are attached respectively to a plurality of livestock animals bred in a stockbreeding facility. This enables the livestock management system 100 to collectively manage a plurality of livestock animals that is bred in a single stockbreeding facility. In addition, this enables the user to check, via the user terminal 4, for example, the astasia-notification information items of the plurality of livestock animals that the user breeds.

Description of this embodiment is made with focus on one of the sensor devices 1.

FIG. 2 is a view illustrating an external appearance of the sensor device 1. FIG. 3 is a view illustrating how the sensor device 1 is attached to a cattle individual as a livestock animal.

The sensor device 1 has a casing 10 configured to be capable of being attached to the head of the livestock animal A. By attaching the casing 10 to the head of the livestock animal A, a posture of the livestock animal A can be accurately detected as described below. Further, the head of the livestock animal herein refers to a distal part with respect to the neck of the livestock animal, which includes parts such as the submental part, the occipital part, the supranasal part, and the like.

The casing 10 has a size and a shape that enable attachment, for example, to the livestock animal being an attachment target. The casing 10 has a waterproof structure, a shockproof structure, or the like as appropriate. A size of a longest part of the casing 10 may be set, for example, to from approximately several cm to several tens of cm.

In the example illustrated in FIG. 2, the casing 10 has a rectangular shape with rounded corners as a whole, and its top surface has a dome shape. By forming the casing 10 into the shape with the rounded corners, stress to the livestock animal at the time of attachment can be alleviated. The casing 10 need not necessarily be formed into the shape illustrated in FIG. 2, and may have, for example, a disc-like (columnar) shape, an elliptical shape, a rectangular parallelepiped shape, a square columnar shape, or shapes similar to these shape.

The casing 10 may be made, for example, of a resin material or the like, which may contain an anti-allergenic material, an antibacterial material, or the like.

As illustrated in FIG. 3, an attachment 17 for enabling the casing 10 to be attached to the head of the livestock animal A may be attached to the casing 10.

The attachment 17 includes a cord 171, which is configured as a bridle to be attached to the head of the livestock animal A. By providing the cord 171 to the attachment 17, a contact area of the livestock animal A and the attachment 17 can be reduced. With this, stress to the livestock animal A can be alleviated.

Further, the cord 171 may be made of a natural material such as hemp, leather, or cotton. With this, stress to the livestock animal A due to the contact of the attachment 17 can be further alleviated.

The attachment 17 is configured to be capable of enabling the casing 10 to be attached to the livestock animal A in a displacement-free manner. The method of holding the casing 10 with the cord 171 is not limited to that of the illustrated example. For example, another cord for fixing the casing 10 to the cord 171 may be used.

Still further, by contriving a knot or the like, the cord 171 can be adjusted to an appropriate length in accordance with a size of the head of the livestock animal A. With this, another member for adjusting the length of the cord 171 may be omitted, and stress to the livestock animal A can be further alleviated.

The casing 10 is configured to be capable of being attached, for example, to the submental part of the livestock animal A. With this, even when the sensor device 1 is attached to the livestock animal A in the displacement-free manner, by weight of the sensor device 1 itself, a small clearance is formed between the livestock animal A and the sensor device 1. Thus, stress to the livestock animal A due to the contact of the casing 10 can be alleviated.

As illustrated in FIG. 1, the relay device 2 receives the astasia-notification data item transmitted from the sensor device 1, and transmits this data item to the server 3 via the network N. In other words, the relay device 2 is configured as a communication device capable of communicating with the sensor device 1, and capable of connecting to the network N. The relay device 2 may include a dedicated communication device, or may include one or a plurality of information processing devices (such as a PC (Personal Computer), a smartphone, and a tablet terminal). Alternatively, the relay device 2 may include both the communication device and the information processing device.

As examples of the network N, there may be mentioned the Internet, a local area network, and the like.

In this embodiment, the relay device 2 includes a reception device 21 capable of communicating with the sensor device 1, and a transmission device 22 capable of being connected to the network N. Details of their configurations and the like are described below.

The relay device 2 is installed, for example, in the stockbreeding facility. In this case, the relay device 2 is installed, for example, in a passage or the stall in the barn, a grazing zone, or an administrative building for the users. Alternatively, the relay device 2 may be installed on an outside of the stockbreeding facility, or may be shared among a plurality of stockbreeding facilities that introduces the livestock management system 100.

In addition, the reception device 21 and the transmission device 22 may be installed close to each other on the same site, or may be installed away from each other on different sites. For example, the reception device 21 may be installed in the barn, and the transmission device 22 may be installed, for example, in the administrative building.

The relay device 2 may include a plurality of reception devices 21. The plurality of reception devices 21 may each be installed for the several stalls, or may be installed respectively in the stalls. In this case, the relay device 2 may include the one transmission device 22, or may include a plurality of transmission devices 22.

The server 3 is an information processing device on the network N. The server 3 may include one information processing device, or may include a plurality of information processing devices.

In this embodiment, the server 3 is a device different from the sensor device 1 and the relay device 2. The server 3 receives the astasia-notification data item transmitted from the relay device 2, and processes and transmits this data item to the user terminal 4. A device information item of the relay device 2, and an information item about, for example, an intensity of the received signal may be added by the relay device 2 to the astasia-notification data item that the server 3 receives.

The server 3 is capable of providing a livestock management service to the user terminal 4 via the network N. For example, the server 3 is capable of providing the livestock management service to the user terminal 4 via livestock-management application software (hereinafter, abbreviated as “livestock management application”).

The server 3 may provide the livestock management application in a form of web application to, for example, the user terminal 4, or may distribute the livestock management application to the user terminal 4, and then cause the user terminal 4 to install this application therein.

The user terminal 4 is an information processing device to be operated by the user who manages the livestock animal A. The user terminal 4 is configured to be capable of communicating with the server 3 on the network N. Examples of the user terminal 4 include a smartphone, a tablet terminal, a PC (Personal Computer), a wearable device, and the like.

In this embodiment, the user terminal 4 has the livestock management application installed therein, and executes processes on the basis of this software.

[Hardware Configurations in Livestock Management System]

FIG. 4 is a block diagram showing respective hardware configurations of the devices included in the livestock management system 100.

(Sensor Device)

The sensor device 1 includes a power supply unit 11, a sensor unit 12, a control unit 13, and a communication unit 14.

The power supply unit 11 includes a battery 111 and a power supply circuit 112.

The battery 111, which supplies power for the sensor device 1, includes a primary battery such as a lithium primary battery, a zinc-air battery, a manganese dry cell, an alkaline dry cell, a silver oxide battery, or the like. Alternatively, the battery 111 may include a secondary battery.

The power supply circuit 112, which includes, for example, an integrated circuit (IC), supplies the power supplied from the battery 111 to the sensor unit 12 as stabilizing power at a predetermined voltage value.

The sensor unit 12 includes an acceleration sensor 121, a first comparator 122, a counter 123, and a second comparator 124.

The acceleration sensor 121, which has a plurality of detection axes, outputs values based on respective accelerations along the detection axes.

The first comparator 122 and the second comparator 124, each of which is a comparator circuit that compares an input value and a threshold to each other, each output a signal when the input value is larger than the threshold. The counter 123 counts the signals output from the first comparator 122 in a predetermined sampling period.

The first comparator 122, the counter 123, and the second comparator 124 function as a noise removing circuit that outputs a detection signal when the output values from the acceleration sensor 121 include output values that are each equal to or larger than corresponding one of preset output values and that are output for a predetermined time period or longer. Specific processes by the sensor unit 12 are described below.

FIG. 5 is a schematic view illustrating a relationship between the detection axes of the acceleration sensor 121 and the head of the livestock animal A under a state in which the sensor device 1 is attached. As illustrated in FIG. 5, the casing 10 of the sensor device 1 is configured to be capable of being attached to the submental part of the livestock animal A.

In this embodiment, the acceleration sensor 121 has an x-axis that is arranged along an anterior-and-posterior direction of the livestock animal, a y-axis that is arranged along a right-and-left direction of the livestock animal, and a z-axis that is arranged along a superior-and-inferior direction of the livestock animal. Typically, these detection axes are orthogonal to each other.

Herein, the anterior-and-posterior direction of the livestock animal refers to a direction parallel to a horizontal direction under a state in which the livestock animal assumes a standing posture, that is, a direction that extends from a front (anterior) at which the face points toward a rear (posterior) at which the tail points.

The right-and-left direction of the livestock animal refers to a right-and-left direction parallel to the horizontal direction under the state in which the livestock animal assumes the standing posture.

The superior-and-inferior direction of the livestock animal refers to a direction parallel to a gravity direction under the state in which the livestock animal assumes the standing posture, that is, a direction that extends from a top (superior) at which the head is present toward a bottom (inferior) at which toes are present.

As shown in FIG. 4, the control unit 13 includes a processor 131, a memory 132, and a timer 133.

The processor 131, which may include, for example, an MPU (Micro Processing Unit), a CPU (Central Processing Unit), or the like, includes the MPU in this embodiment. With this, the sensor device 1 can be downsized.

The memory 132, which typically includes a ROM (Read Only Memory), a RAM (Random Access Memory), and the like, may store identification information items for identifying the livestock animals. The identification information items of the livestock animals are not particularly limited as long as the sensor devices 1 or the livestock animals wearing these sensor devices 1 can be identified. For example, identifiers (IDS) unique to the sensor devices 1, individual identification numbers of the livestock animals, or the like may be used. The identification information items of the livestock animals may include any one or both of these information items. Also when the identification information items of the livestock animals include only the identifiers unique to the sensor devices 1, the sensor devices 1 and the livestock animals wearing these sensor devices 1 correspond one by one to each other. With this, the livestock animals can be identified. Further, the memory 132 may store attachment start dates of the sensor devices 1. The attachment start dates are stored, for example, by activating the sensor devices 1 at the time of attachment to the livestock animals.

The timer 133 is capable of measuring time periods, and when to start and stop the timekeeping is controlled by the processor 131.

The communication unit 14 includes a communication circuit 141 and an antenna 142.

The communication circuit 141, which includes, for example, by a radio-frequency integrated circuit (RF-IC), is capable of executing signal processes for the transmission. In this embodiment, the communication circuit 141 is capable of executing processes for wireless communication. As examples of the wireless communication, there may be mentioned a communication type utilizing electromagnetic waves or infrared rays, a communication type utilizing an electric field, a communication type utilizing acoustic waves, and the like. As examples of specific schemes of the communication types, there may be mentioned a communication scheme that utilizes electromagnetic waves in a band of 920 MHz. In addition, there may be employed other communication schemes utilizing electromagnetic waves in a band of from several hundreds MHz (megahertz) to several GHz (gigahertz), such as “Wi-Fi (trademark),” “ZigBee (trademark),” “Bluetooth (trademark),” “Bluetooth Low Energy,” “ANT (trademark),” “ANT+ (trademark),” and “EnOcean (trademark).”

The antenna 142 is capable of performing wireless communication with the relay device 2.

(Relay Device)

As described above, the relay device 2 includes the reception device 21 and the transmission device 22.

The reception device 21 is configured to be capable of communicating with the sensor device 1. The reception device 21 includes, for example, a communication circuit that executes communication processes, an antenna, and a control circuit that performs, for example, control of the communication circuit (none of which is shown). The reception device 21 may, for example, be a dedicated communication device, or may be a dedicated information-processing device.

The reception device 21 is configured to be capable of performing the wireless communication according, for example, to the communication scheme utilizing electromagnetic waves or infrared rays, the communication scheme utilizing an electric field, or the communication scheme utilizing acoustic waves. The reception device 21 need not necessarily be capable of performing only the wireless communication, and may be capable of performing wired communication.

The transmission device 22 is configured to be capable of being connected to the reception device 21 and connected to the network N. The transmission device 22 includes, for example, a communication circuit, an antenna, and a control circuit (none of which is shown). The transmission device 22 may, for example, be a dedicated communication device, or may be a dedicated information-processing device.

Examples of a communication scheme adaptable to the transmission device 22 include communication schemes that enable establishment of the connection to the network N, more specifically, communication schemes using a wireless LAN (according, for example, to IEEE 802.11) such as Wi-Fi (trademark), using a wired LAN, or using a 3G or a 4G network for mobile communication. When the communication methods such as Wi-Fi are adopted to the transmission device 22, the transmission device 22 can be connected to the network N via a predetermined access point.

The transmission device 22 may be connected to the reception device 21, for example, in a wired manner via a cable or the like, or in a wireless manner.

(Server)

The server 3 includes a control unit 31, a storage unit 32, and a communication unit 33.

The control unit 31, which includes a processor and memories including the ROM and the RAM (none of which is shown), collectively controls all the units in the server 3. The processor includes the CPU. The ROM stores programs to be executed by the processor. The RAM is used, for example, as a working memory at the time when the processor executes the processes. The control unit 31 executes predetermined processes in accordance, for example, with the control programs stored in the memory.

The storage unit 32, which is configured, for example, as a storage of the server 3, includes nonvolatile memories such as an HDD (Hard Disk Drive) and a flash memory (SSD: Solid State Drive).

In this embodiment, the storage unit 32 may store user information items of the users, and sensor information items of the sensor devices 1 attached to the livestock animals that the users manage.

The user information items may each include, for example, an identification information item of the user terminal 4 (such as device token, registration ID in the livestock management service, or terminal ID), personal information items of the user (such as his/her name, name of his/her stockbreeding facility, and location of the stockbreeding facility), and the like. The user information items may each, for example, be an information item input 4 by the user via the user terminal, or be an information item issued by the server 3 at a time of a process of authenticating the user terminal 4.

The sensor information items may each include, for example, the identification information item of the livestock animal (such as identifier of the sensor device 1, an individual-identification information item of the livestock animal, or the like), information items about the attachment start date of corresponding one of the sensor devices 1, a breeding site of corresponding one of the livestock animals, a radio wave condition of the sensor device 1, a remaining capacity of the battery of the sensor device 1, and the like. The sensor information item of each of the sensor devices 1 may, for example, be an information item input by the user via the user terminal 4, or be an information item transmitted from the sensor device 1 and received by the server 3. The sensor information item is stored in correspondence with the user information item of the user who manages the livestock animal to which the sensor device 1 is attached.

The communication unit 33 is configured to be capable of being connected to the network N, and capable of communicating with the relay device 2 and the user terminal 4. The communication unit 33 can be connected to the network N via hardware network interfaces for the wireless LAN (according, for example, to IEEE802.11) such as Wi-Fi (trademark), and the wired LAN.

The server 3 may include not only the above-described components but also components such as a display unit and an input operation unit as appropriate.

(User Terminal)

The user terminal 4 includes a control unit 41, a storage unit 42, a communication unit 43, a display unit 44, and an input operation unit 45.

The control unit 41 includes a processor that includes the CPU, and the memories such as the ROM and the RAM, and collectively controls all the units in the user terminal 4. The control unit 41 executes predetermined processes in accordance with control programs stored in the memories.

The storage unit 42, which is configured as a storage of the user terminal 4, includes the nonvolatile memory and the like. The storage unit 42 may store some of the user information items and the sensor information items stored in the storage unit 32 of the server 3.

The communication unit 43 is configured to be capable of being connected to the network N, and capable of communicating with the server 3. Specifically, the communication unit 43 can be connected to the network N by using the wireless LAN (according, for example, to IEEE802.11) such as Wi-Fi (trademark), or by using the 3G or the 4G network for mobile communication. This enables the communication unit 43 to communicate with the server 3.

The display unit 44 includes a display device such as an LCD (Liquid Crystal Display) and an organic EL (Electroluminescence) panel. The display unit 44 may include not only the display device but also a D/A conversion circuit and the like.

The input operation unit 45 is, for example, a touchscreen, a keyboard, a pointing device such as a mouse, or other input devices. When the input operation unit 45 is the touchscreen, the touchscreen can be integrated with the display unit 44.

Note that, the user terminal 4 may include not only the above-described components but also a battery, a camera, a microphone, a speaker, and the like (none of which is shown).

[Functional Configuration of Livestock Management System]

FIG. 6 is a block diagram showing a functional configuration of the livestock management system 100.

In this embodiment, the sensor device 1 includes a detection unit 101, a postural-state determination unit 102, a state inference unit 103, a transmission unit 104, and the casing 10 that houses the detection unit 101, the postural-state determination unit 102, the state inference unit 103, and the transmission unit 104.

In this embodiment, the relay device 2 includes a relay unit 105.

In this embodiment, the server 3 includes a notification-information generation unit 106.

In this embodiment, the user terminal 4 includes a notification unit 107.

The detection unit 101 outputs, to the postural-state determination unit 102, the detection signals each including corresponding ones of the output values from the acceleration sensor 121. The detection unit 101 can correspond to the sensor unit 12.

The detection unit 101 outputs the detection signal, for example, when the output values from the acceleration sensor 121 include the output values that are each larger than corresponding one of the preset output values and that continue to be detected. Specifically, the detection unit 101 is capable of outputting the detection signal when the output values from the acceleration sensor 121 include output values that are each larger than corresponding one of the preset output values and that continue to be detected for a predetermined state-determination time period or longer. This state-determination time period is not particularly limited as long as states in which the livestock animal does not maintain predetermined postures can be excluded. For example, this state-determination time period may be set to several seconds or more and less than several minutes.

With this, the processes can be executed while excluding the states in which the livestock animal acts without maintaining the predetermined postures.

The output values on the basis of which the detection unit 101 outputs the detection signal may correspond to each of the detection axes of the acceleration sensor 121. For example, The output values on the basis of which the detection unit 101 outputs the detection signal may correspond to any one of a prostration detection axis and a non-prostration detection axis of the acceleration sensor 121. With this, as described below, postural states can be detected on the basis of values of gravitational accelerations, which are detected respectively along these detection axes.

The prostration detection axis, which corresponds to one of the detection axes of the acceleration sensor 121, enables detection of a highest acceleration in the gravity direction from among the accelerations along the plurality of detection axes in the prostrate state of the livestock animal. The prostration detection axis is defined, for example, as the y-axis shown in FIG. 5.

The non-prostration detection axis, which corresponds to one of the detection axes of the acceleration sensor 121, enables detection of the highest acceleration in the gravity direction from among the accelerations along the plurality of detection axes in the non-prostrate state of the livestock animal. The non-prostration detection axis is defined, for example, as the z-axis shown in FIG. 5.

Now, the postural states of the livestock animal are described. The postural states of the livestock animal each refers to a state in which the livestock animal maintains a predetermined posture, which include, for example, a prostrate state and a non-prostrate state.

The prostrate state of the livestock animal refers to a state in which the livestock animal maintains a lying posture (prostrate posture) in which the livestock animal lets its head and trunk laid laterally, and lets its legs stretched out along a substantially horizontal direction.

The non-prostrate state of the livestock animal refers to states other than the prostrate state, such as a state in which the livestock animal maintains a standing posture, and a non-prostrate posture such as a prone posture in which the livestock animal lets its chest up with its legs being tucked.

Next, with reference to A and B of FIG. 7, the postures of the livestock animal A and values of the accelerations along the detection axes are described. Note that, in FIG. 7, a Y-axis direction and a Z-axis direction are defined as biaxial directions orthogonal to each other in an absolute coordinate system, and the Y-axis direction corresponds to the horizontal direction, and the Z-axis direction corresponds to the gravity direction. In addition, the y-axis direction and the z-axis direction are defined as biaxial directions orthogonal to each other in a relative coordinate system that belongs to the sensor device 1. As in FIG. 5, the y-axis direction is identical to the right-and-left direction of the livestock animal A, and the z-axis direction is identical to the superior-and-inferior direction of the livestock animal A.

A of FIG. 7 is a schematic view illustrating the sensor device 1 under a state in which the livestock animal wearing the sensor device 1 has been in the prostrate posture.

The prostrate posture of the livestock animal A may be defined, for example, as a posture in which the z-axis direction identical to the superior-and-inferior direction of the livestock animal A has turned at an angle θ₁₁ [°] higher than an angle θ₁₀ [°] from the Z-axis direction being the gravity direction. θ₁₀ [°] may be set, for example, to 30° or more and 90° or less, or, for example, to 40° or more and 70° or less.

Note that, the z-axis that has turned at θ₁₀ [°] from the Z-axis is defined as a z′-axis, and the y that has turned at θ₁₀ [°] from the Y-axis is defined as a y′-axis.

In the case of the prostrate posture illustrated in A of FIG. 7, a component Gy₁₁ in the y-axis direction of a gravitational acceleration g[m/s²] is sin(θ₁₁·π/180)·g[m/s²], a value of which is large than that of sin(θ₁₀·π/180)·g[m/s²] being a component Gy₁₀ in the y′-axis direction of the gravitational acceleration g[m/s2].

Thus, detection that the livestock animal A has been in the prostrate posture can be performed by setting corresponding one of the preset output values with respect to output values corresponding to the y-axis of the acceleration sensor 121 to a value corresponding to sin(θ₁₀·π/180)·g[m/s²].

B of FIG. 7 is a schematic view illustrating the sensor device 1 under a state in which the livestock animal wearing the sensor device 1 has been in the non-prostrate posture.

The non-prostrate posture of the livestock animal A may be defined, for example, as a posture in which the z-axis direction has turned at an angle θ₂₁ [°] lower than an angle θ₂₀ [°] from the Z-axis direction. θ₂₀ [°] may be set, for example, to 0° or more and 60° or less, or, for example, to 20° or more and 50° or less. Alternatively, θ₂₀ [°] may be defined, for example, as a turning angle as viewed from the Z-axis direction at a time when the z-axis direction has turned at a predetermined angle from θ₁₁ [°] under the state in which the livestock animal has been in the prostrate posture toward the Z-axis direction. The predetermined angle may be set, for example, to 5° or more and 20° or less.

Note that, the z-axis that has turned at θ₂₀ [°] from the Z-axis is defined as a z″-axis.

In the case of the non-prostrate posture illustrated in B of FIG. 7, a component Gz₂₁ in the z-axis direction of the gravitational acceleration g[m/s²] is cos(θ₂₁·π/180)·g[m/s²], a value of which is large than that of cos(θ₂₀·π/180)·g[m/s²] being a component Gz₂₀ in the z″-axis direction of the gravitational acceleration g[m/s²].

Thus, detection that the livestock animal A has been in the non-prostrate posture can be performed by setting another corresponding one of the preset output values with respect to output values corresponding to the z-axis of the acceleration sensor 121 to a value corresponding to cos(θ₂₀·π/180)·g[m/s²].

Further, in this embodiment, by attaching the casing 10 to the head of the livestock animal A, as described below, a correlation between the output values from the acceleration sensor 121 and the postures of the livestock animal A can be increased. With this, the prostrate posture of the livestock animal A can be accurately detected.

For example, when the casing 10 is attached to the leg of the livestock animal A with a belt or the like, the sensor device 1 may unexpectedly move along a circumferential direction of the leg by walking movement or the like of the livestock animal A. As a result, the x-axis and the y-axis of the acceleration sensor 121 may be disturbed with respect to the livestock animal A. As a countermeasure, the casing 10 is attached to the head of the livestock animal A. With this, the detection axes of the acceleration sensor 121 with respect to the livestock animal A that can assume various postures can be further stably arranged.

In addition, when the casing 10 is attached to a part other than the head of the livestock animal A, such as the leg, output values in the prostrate posture and output values in the prone posture being the non-prostrate posture are unlikely to differ from each other. As a result, the prone posture may be falsely detected as the prostrate posture. By attaching the casing 10 to the head of the livestock animal A, the prostrate posture in which the head is laid perfectly laterally can be accurately detected from the output values on the basis of the acceleration sensor 121.

The detection unit 101 is capable of detecting, by processes as follows in the sensor unit 12, that the output values from the acceleration sensor 121 include the output values that are each larger than corresponding one of the preset output values and that are output for the predetermined state-determination time period or longer.

First, when the output values from the acceleration sensor 121 are each larger than the preset output value, the first comparator 122 outputs these output values to the counter 123.

The counter 123 counts the output from the first comparator 122 in the predetermined sampling period.

When the count value from the counter 123 becomes larger than a preset count value, the second comparator 124 outputs a processing result as the detection signal to the control unit 13 (processor 131). The preset count value is, for example, a value to be calculated by dividing the state-determination time period by the sampling period of the counter 123.

In this way, the second comparator 124 can output the detection signals when a time period of the output from the first comparator 122 is longer than the state-determination time period.

The detection signals that the detection unit 101 transmits may include information items about the detection axes of the acceleration sensor 121, which are used in the processes.

The detection signals may each be a signal that requests an interruption procedure into the control unit 13, which may include, for example, a flag indicating the request for the interruption procedure.

The postural-state determination unit 102 determines in which of the prostrate state and the non-prostrate state the livestock animal has been on the basis of the output values from the acceleration sensor 121. The postural-state determination unit 102 can correspond to the control unit 13.

The postural-state determination unit 102 is capable of determining in which of the prostrate state and the non-prostrate state the livestock animal has been on the basis of, for example, the detection signals from the detection unit 101. By using these detection signals, such a case where the livestock animal immediately varies its posture can be excluded, and in which of the states the livestock animal has been can be accurately determined.

In addition, the postural-state determination unit 102 is capable of executing the state-determination process with reference only to the output values that correspond to corresponding one of the detection axes. Specifically, the postural-state determination unit 102 is capable of determining that the livestock animal has been in the prostrate state on the basis of ones of the output values from the acceleration sensor 121, which correspond to the prostration detection axis, and capable of determining that the livestock animal has been in the non-prostrate state on the basis of other ones of the output values from the acceleration sensor 121, which correspond to the non-prostration detection axis.

In this embodiment, the postural-state determination unit 102 is capable of determining that the livestock animal has been in the prostrate state on the basis of the detection signal based on the ones of the output values, which correspond to the prostration detection axis, and capable of determining that the livestock animal has been in the non-prostrate state on the basis of the detection signal based on the other ones of the output value, which correspond to the non-prostration detection axis.

With this, the number of processes in the control unit 13 can be reduced, which can contribute to downsizing of the device.

In addition, the postural-state determination unit 102 may include a plurality of determination modes in each of which one of the postural states is detected on the basis of the output values corresponding to only one of the detection axes. Specifically, the postural-state determination unit 102 may include a prostrate-state determination mode in which the prostrate state can be detected on the basis of the ones of the output values, which correspond to the prostration detection axis, and include a non-prostrate-state determination mode in which the non-prostrate state can be detected on the basis of the other ones of the output values, which correspond to the non-prostration detection axis. In this case, after the postural-state determination unit 102 has determined that the livestock animal has been in the prostrate state in the prostrate-state determination mode, the postural-state determination unit 102 can be switched into the non-prostrate-state determination mode. Similarly, after the postural-state determination unit 102 has determined that the livestock animal has been in the non-prostrate state in the non-prostrate-state determination mode, the postural-state determination unit 102 can be switched into the prostrate-state determination mode. Specifically, this mode switching of the postural-state determination unit 102 can be performed by the processor 131 of the control unit 13.

In this embodiment, the prostrate-state determination mode is a determination mode in which the prostrate state can be detected on the basis of the detection signal based on the ones of the output values, which correspond to the prostration detection axis. Similarly, the non-prostrate-state determination mode is a determination mode in which the non-prostrate state can be detected on the basis of the detection signal based on the other ones of the output values, which correspond to the non-prostration detection axis.

With this, the output values to be referred to at the time of determining in which of the states the livestock animal has been can be automatically selected in accordance with the mode switching. As a result, the number of processes in the control unit 13 can be further reduced.

In addition, also when the postural-state determination unit 102 includes the above-described determination modes, the postural-state determination unit 102 is capable of determining that in which of the postural states the livestock animal has been on the basis of the detection signals. For example, the postural-state determination unit 102 is capable of permitting an interruption procedure based on the detection signal corresponding to the y-axis being corresponding one of the detection axes of the acceleration sensor 121 in the prostrate-state determination mode, and capable of permitting an interruption procedure based on the detection signal corresponding to the z-axis being corresponding one of the detection axes of the acceleration sensor 121 in the non-prostrate-state determination mode.

The state inference unit 103 infers whether or not the livestock animal has developed the astasia on the basis of a duration of the prostrate state. The state inference unit 103 can correspond to the control unit 13.

In this embodiment, the state inference unit 103 infers that the livestock animal has developed the astasia when the prostrate state has continued for a predetermined duration or longer. Specifically, after the postural-state determination unit 102 has determined that the livestock animal has been in the prostrate state, the state inference unit 103 activates the timer 133, and infers that the livestock animal has developed the astasia when the prostrate state has continued for the predetermined duration or longer on the basis of the time period measured by the timer 133.

For example, the state inference unit 103 may infer that the livestock animal has developed the astasia when the postural-state determination unit 102 does not determine that the livestock animal has been in the non-prostrate state in the duration. When it is determined that the livestock animal A has been in the non-prostrate state, the state inference unit 103 stops the timer 133, and ends the inference procedure.

The duration as a reference for the determination by the state inference unit 103 may be set, for example, to 10 minutes or more, or desirably to 20 minutes or more.

FIG. 8 is a chart showing results of an experiment for investigating the duration, which was carried out with respect to four livestock animals. In this experiment, the sensor devices 1 were attached to the heads of the four livestock animals, and respective durations of the prostrate states that occurred a total of 572 times in approximately two weeks were measured.

In this experiment, as shown in FIG. 8, the number of times of prostrate states that continued 10 minutes or more and less than 20 minutes was only 1.7%. Therefore, it is conceivable that, by setting the duration to 10 minutes or more, the users can be reliably notified of livestock animals that are highly likely to develop the astasia.

Further, there were no livestock animals that continued to be in the prostrate state for 20 minutes or more, and all the livestock animals restored to the non-prostrate state within 20 minutes. These results demonstrated that the livestock animals were highly likely to develop the astasia when the duration of the prostrate state was 20 minutes or more. Therefore, it is conceivable that, by setting the duration, for example, to 20 minutes or more, the inference that the livestock animal has developed the astasia can be made further accurately.

The state inference unit 103 may infer that the astasia is urgent depending on the duration of the prostrate state. For example, the state inference unit 103 may infer that first astasia has developed when the prostrate state has continued for a first duration or longer, and may infer that second astasia that is more urgent than the first astasia has developed when the prostrate state has continued for a second duration that is longer than the first duration, or longer. The first astasia may be defined, for example, as a state in which the livestock animal is likely to develop the astasia, and the second astasia may be defined, for example, as a state in which the livestock animal is highly likely to have already developed the astasia. With reference to the experimental data items shown in FIG. 8, as specific durations, the first duration is set, for example, to from 10 minutes or more and less than 20 minutes, and the second duration is set, for example, to 20 minutes or more. With this, it is conceivable that the users can be accurately notified of the information items about the astasia of the livestock animals.

In this way, the users can be notified of the information items about the astasia of the livestock animals in a stepwise manner. As a result, the state of the livestock animal can be further accurately notified of, and treatment can be further effectively provided.

The state inference unit 103 is capable of storing time points and dates when the inferences that the astasia has developed are made. This enables the users to be notified, for example, of the time points when the inferences that the astasia has developed are made.

When the inference that the livestock animal has developed the astasia is made, the transmission unit 104 transmits, to the server 3, the astasia-notification data item indicating that the inference that the astasia has developed is made. The transmission unit 104 can correspond, for example, to the control unit 13 and the communication unit 14.

The astasia-notification data item may include, for example, an information item indicating that the inference that the astasia has developed is made, an information item about the time point and the date when the inference that the astasia has developed is made, and an identification information item of a livestock animal to which the inference that the astasia has developed is made. The information item indicating that the inference that the astasia has developed is made may include, for example, a flag indicating that the inference that the astasia has developed is made. The astasia-notification data item may include not only these information items but also, for example, the output values from the acceleration sensor 121, which are used in the process of determining whether or not the livestock animal has been in the prostrate state, the remaining capacity of the battery of the sensor device 1, the radio wave condition, and other information items that the sensor device 1 stores.

As the identification information item of the livestock animal, for example, the identifier (ID) unique to the sensor device 1 or the like may be used. This identifier may be allocated in advance, for example, to the sensor device 1, or may be allocated each time. For example, when the sensor device 1 establishes communication connection with, for example, the relay device 2, the identifier may be allocated, and the allocated identifier may be used. Alternatively, the identification information item of the livestock animal may include, instead of the identifier or in addition to the identifier, for example, the individual identification number of the livestock animal.

In addition, the transmission unit 104 is capable of transmitting a first-astasia notification data item to the server 3 when the state inference unit 103 infers that the first astasia has developed. The transmission unit 104 is capable of transmitting a second-astasia notification data item to the server 3 when the state inference unit 103 infers that the second astasia has developed.

The first-astasia notification data item is a data item indicating that the inference that the first astasia has developed is made. This first-astasia notification data item may include, for example, an information item indicating that the inference that the first astasia has developed is made, an information item about a date and a time point when the inference that the first astasia has developed is made, and the identification information item of the livestock animal. Specifically, the information item indicating that the inference that the first astasia has developed is made may include a flag indicating the first astasia.

The second-astasia notification data item is a data item indicating that the inference that the second astasia has developed is made. This second-astasia notification data item may include an information item indicating that the inference that the second astasia has developed is made, an information item about a date and a time point when the inference that the second astasia has developed is made, and the identification information item of the livestock animal. Specifically, the information item indicating that the inference that the second astasia has developed is made may include a flag indicating the second astasia. A flag that is used as the flag indicating the second astasia may be different from the flag indicating the first astasia.

In this way, the transmission unit 104 can transmit the astasia-notification data items that are different in urgency from each other.

Further, when the inference that the astasia has developed is made, and then when the postural-state determination unit 102 determines that the livestock animal has been in the non-prostrate state, the transmission unit 104 may transmit an astasia-clearance notification data item to the server 3. The astasia-clearance notification data item may include, for example, an information item indicating that the astasia has been cleared, an information item about a time point and a date when the astasia has been cleared, and an identification information item of a livestock animal from which the astasia has been cleared. The information item indicating that the astasia has been cleared may include, for example, a flag indicating that the astasia has been cleared.

In this way, the users can be notified that the astasia has been cleared.

Still further, in a case where a process of the transmission to the server 3 fails, the transmission unit 104 may execute a retry process. The preset number of the retry processes is not limited, and settings may be made such that the retry process is unlimitedly executed until the transmission process is successfully executed. With this, the information items about the notification data items can be reliably transmitted to the server 3.

The relay unit 105 relays the notification data items transmitted from the sensor device 1, such as the astasia-notification data items. The relay unit 105 can correspond to the reception device 21 and the transmission device 22 of the relay device 2.

The notification-information generation unit 106 generates, when the inference that the livestock animal has developed the astasia is made, the astasia-notification information item including the astasia-notification data item indicating that the inference that the astasia has developed is made. The notification-information generation unit 106 can correspond, for example, to the control unit 31 of the server 3.

In this embodiment, the notification-information generation unit 106 generates the astasia-notification information item including the astasia-notification data item transmitted from the transmission unit 104. The astasia-notification information item, which includes an information item about the astasia-notification data item, may further include the sensor information item of the sensor device 1, which relates to the astasia-notification data item, an information item about an image to be displayed on the user terminal 4, and the like.

In addition, when the astasia-clearance notification data item is transmitted from the transmission unit 104, the notification-information generation unit 106 may generate an astasia-clearance notification information item including the astasia-clearance notification data item.

The notification unit 107 notifies the user of the astasia-notification information item. The notification unit 107 can correspond, for example, to the control unit 41 and the display unit 44 of the user terminal 4.

The notification unit 107 is capable of notifying the user of the astasia-notification information item generated by the server 3, for example, by causing the display unit 44 to display this astasia-notification information item. Alternatively, the notification unit 107 may notify of the astasia-notification information item by voice via a speaker or the like, or may notify of the astasia-notification information item in the form of vibration. The notification may be issued by combining these plurality of notification methods with each other.

In addition, the notification unit 107 is capable of notifying the user of the astasia-clearance notification information item when the server 3 generates the astasia-clearance notification information item.

[Operation Examples of Sensor Device]

FIG. 9 and FIG. 10 are flowcharts showing operation examples of the sensor device 1. With reference to these drawings, an operation example in which the sensor device 1 executes a detection-signal output procedure of outputting the detection signal, and an operation example in which the sensor device 1 executes the astasia inference procedure of inferring, on the basis of this detection signal, whether or not the livestock animal has developed the astasia are described.

The livestock animal in these operation examples is a cattle individual.

As illustrated in FIG. 5, the sensor device 1 is attached to the submental part of the livestock animal A. The prostration detection axis of the acceleration sensor 121 is identical to the y-axis along the right-and-left direction of the livestock animal A, and the non-prostration detection axis of the acceleration sensor 121 is identical to the z-axis along the superior-and-inferior direction of the livestock animal A.

In addition, by a time point when the procedure is started, the counter 123 has already been cleared, and the timer 133 has already been stopped.

(Detection-Signal Output Procedure)

FIG. 9 is a flowchart showing the operation example of the detection-signal output procedure by the sensor unit 12 (detection unit 101).

First, the first comparator 122 compares each of the output values from the acceleration sensor 121 and corresponding one of the preset output values to each other (S101). When all these output values are larger than the preset output value (YES in S101), the first comparator 122 outputs an output signal.

In this operation example, the output values from the acceleration sensor 121 may be the output values corresponding to one of the y-axis and the z-axis.

Further, as an example of the preset output value corresponding to the y-axis, there may be mentioned an output value corresponding to an acceleration value (sin(θ₁₀·π/180)·g[m/s²]) at a time when θ₁₀ [°] in A of FIG. 7 is 40° or more and 70° or less. As an example of the output value corresponding to the z-axis, there may be mentioned an output value corresponding to an acceleration value (cos(θ₂₀·π/180)·g[m/s²]) at a time when θ₂₀ [°] in B of FIG. 7 is 20° or more and 50° or less.

The counter 123 performs count-up on the basis of the output signals from the first comparator 122 (S102). The counter 123 counts up the output signals from the first comparator 122 at a predetermined sampling interval. The counter 123 continues the count-up while the comparator 122 successively outputs the output signals.

Meanwhile, when the output value from the acceleration sensor 121 becomes smaller than the preset output value (NO in S101), the first comparator 122 stops outputting the output signal, and the counter 123 does not receive the output signal. In this case, the counter 123 clears the count value (S103), and the procedure is ended.

The second comparator 124 compares the count value from the counter 123 and the preset count value to each other (S104). When the count value becomes larger than the preset count value (YES in S104), the second comparator 124 outputs the detection signal (S105). The detection signal, which includes the information item about the detection axis and the interruption flag, is transmitted to the control unit 13. The preset count value is a value calculated by dividing the predetermined state-determination time period by the sampling period of the counter 123. The state-determination time period may be set, for example, to from several seconds to several minutes.

By the above-described procedure, when the output values from the acceleration sensor 121 include the output values that are each larger than corresponding one of the preset output values and that are detected for the predetermined state-determination time period or longer, the sensor unit 12 can output the detection signal based on the output values corresponding to corresponding one of the detection axes.

The detection signal based on the output values corresponding to the y-axis is output when the livestock animal A maintains the prostrate posture for the predetermined state-determination time period or longer.

The detection signal based on the output values corresponding to the z-axis is output when the livestock animal A maintains the non-prostrate posture for the predetermined state-determination time period or longer.

The control unit 13 is capable of executing, after receiving the detection signal, an interruption procedure as follows on the basis of the detection signal.

(Astasia Inference Procedure)

FIG. 10 is a flowchart showing the operation example of the astasia inference procedure by the control unit 13 (postural-state determination unit 102, state inference unit 103, and transmission unit 104).

First, the control unit 13 receives the detection signal from the sensor unit 12 (S201). The astasia inference procedure of this operation example is defined as the interruption procedure based on the detection signal.

In this operation example, the control unit 13 permits the interruption based on the detection signal corresponding to a preset one of the determination modes. Specifically, when the prostrate-state determination mode has been set, the control unit 13 permits interruption based on the detection signal corresponding to the y-axis. When the non-prostrate-state determination mode has been set, the control unit 13 permits interruption based on the detection signal corresponding to the z-axis.

Note that, when the detection signal corresponding to the preset mode is received during the following processes, the procedure is interrupted, and returns to S201.

Then, the control unit 13 determines whether or not the prostrate-state determination mode has been set (S202). When the control unit 13 determines that the prostrate-state determination mode has been set (YES in S202), the detection signal has been output on the basis of the output values corresponding to the y-axis. Thus, the control unit 13 determines that the livestock animal A has been in the prostrate state (S203).

Next, the control unit 13 is switched from the prostrate-state determination mode into the non-prostrate-state determination mode (S204). With this, when the interruption based on the detection signal output on the basis of the output values corresponding to the z-axis occurs during the following processes, the procedure is interrupted, and returns to S201. Note that, the control unit 13 may cancel the prostrate-state determination mode once before or after determining that the livestock animal A has been in the prostrate state (S203), and then may be switched into the non-prostrate-state determination mode.

After that, the control unit 13 activates the timer 133 (S205), and determines whether or not the time period measured by the timer 133 is equal to or longer than the first duration (S206). When the control unit 13 determines that the time period is equal to or longer than the first duration (YES in S206), the control unit 13 infers that the first astasia has developed (S207). At this time, the control unit 13 may store, for example, the time point when the inference that the first astasia has developed is made. Then, the control unit 13 generates the first-astasia notification data item indicating that the inference that the first astasia has developed is made, and causes the communication unit 14 to execute the communication process (S208).

The first-astasia notification data item includes, for example, the flag indicating that the inference that the first astasia has developed is made, and the information item about the date and the time point when the inference that the first astasia has developed is made, and the identification information item of the livestock animal.

Even during the processes of inferring that the first astasia has developed (S207 and S208), the control unit 13 continues timekeeping with the timer 133 unless the interruption from the sensor unit 12 occurs. Then, the control unit 13 determines whether or not the time period measured by the timer 133 is equal to or longer than the second duration (S209). When the control unit 13 determines that the time period is equal to or longer than the second duration (YES in S209), the control unit 13 infers that the second astasia has developed (S210), and stops the timer 133 (S211). The control unit 13 may store, for example, the time point when the inference that the second astasia has developed is made.

Next, the control unit 13 generates a second-astasia notification data item indicating that the inference that the second astasia has developed is made, causes the communication unit 14 to execute the communication process (S212), and then ends the procedure.

The second-astasia notification data item includes, for example, the flag indicating that the inference that the second astasia has developed is made, and the information item about the date and the time point when the inference that the second astasia has developed is made, and the identification information item of the livestock animal.

Meanwhile, when the control unit 13 determines that the non-prostrate-state determination mode has been set (NO in S202), the detection signal has been output on the basis of the output values corresponding to the z-axis. Thus, the control unit 13 determines that the livestock animal A has been in the non-prostrate state (S213).

Then, the control unit 13 is switched from the non-prostrate-state determination mode into the prostrate-state determination mode (S214). Note that, the control unit 13 may cancel the non-prostrate-state determination mode once before or after determining that the livestock animal A has been in the non-prostrate state (S213), and then may be switched into the prostrate-state determination mode.

Next, when the timer 133 has been activated, the control unit 13 stops the timer 133 (S215).

When the control unit 13 has inferred that the first astasia has developed (YES in S216), the control unit 13 generates the astasia-clearance notification data item indicating that the astasia has been cleared. Then, the control unit 13 causes the communication unit 14 to execute the communication process (S217), and ends the procedure.

The astasia-clearance notification data item includes, for example, the flag indicating that the astasia has been cleared, and the information item about the date and the time point when the astasia is cleared, and the identification information item of the livestock animal.

Meanwhile, when the control unit 13 has not inferred that the first astasia has developed (NO in S216), the control unit 13 ends the procedure without performing, for example, generation of the data items.

As described above, the sensor device 1 is capable of determining in which of the prostrate state and the non-prostrate state the livestock animal A has been, and capable of inferring whether or not the astasia has developed on the basis of the durations of the prostrate state. With this, the inference that the livestock animal A has developed the astasia can be properly made.

In addition, by making the inference as to whether or not the astasia has developed in the two steps on the basis of the durations of the prostrate state, the user can be further reliably notified that the livestock animal A is likely to have developed the astasia.

Next, the astasia notification procedure of notifying the user that the inference that the astasia has developed is made is described as an operation in the livestock management system 100.

[Operation Example of Livestock Management System]

FIG. 11 is a flowchart showing an operation example of the astasia notification procedure in the livestock management system.

In FIG. 11, a process of S301 is executed by the sensor device 1, processes of S302 and S303 are executed by the relay device 2, processes of S304 to S306 are executed by the server 3, and processes of S307 and S308 are executed by the user terminal 4.

Note that, before the processes in this operation example, the livestock management application has been installed in the user terminal 4. In addition, the server 3 has stored the user information item, the sensor information item of the sensor device 1 attached to the livestock animal that the user manages, and the like in advance by the process of authenticating the user terminal 4, acceptation of the information items via the user terminal 4, and the like.

First, the sensor device 1 transmits the notification data item (S301). As the notification data item in this operation example, there may be mentioned any one of the first-astasia notification data item, the second-astasia notification data item, and the astasia-clearance notification data item. Note that, as described above, in the case where the transmission process fails, the sensor device 1 may execute the retry process.

Then, the relay device 2 receives the notification data item (S302), and transmits this notification data item to the server 3 (S303).

Next, the server 3 receives the notification data item (S304), and causes the control unit 31 to generate the notification information item including the notification data item (S305). The notification information item, which includes at least the information item about the notification data item, may further include the sensor information item of the sensor device 1, which relates to this notification data item, the information item about the image to be displayed on the user terminal 4, and the like. The storage unit 32 of the server 3 may store the generated notification-information item.

The notification information item at the time when the notification data item is the first-astasia notification data item is defined as the first-astasia notification information item. Similarly, the notification information item at the time when the notification data item is the second-astasia notification data item is defined as the second-astasia notification information item. The notification information item at the time when the notification data item is the astasia-clearance notification data item is defined as the astasia-clearance notification information item.

The server 3 transmits the generated notification-information item to the user terminal 4 (S306). The server 3 is capable of distributing this notification information item, for example, as a push notification to the user terminal 4. This enables the user to be further reliably notified of the notification information item.

Then, the user terminal 4 receives the notification information item (S307), and causes the display unit 44 to display this notification information item (S308). Then, the above-described notification procedure is ended.

FIG. 12 to FIG. 14 are each an exemplary view showing a screen of the livestock management application, which is displayed on the display unit 44 of the user terminal 4.

FIG. 12 shows a sensor-information presentation screen 441 at a time when the notification information item has not been notified of.

The sensor-information presentation screen 441 includes sensor information fields S each including the sensor information item of corresponding one of the sensor devices 1. The sensor information fields S each include, as the sensor information item, the information items about the identifier of the sensor device 1, the attachment start date of the sensor device 1, the breeding site of the livestock animal wearing the sensor device 1 in the stockbreeding facility, the identification number of the livestock animal wearing the sensor device 1, the radio wave condition of the sensor device 1, the remaining capacity of the battery of the sensor device 1, and the like.

The sensor information fields S may each include not only the sensor information item but also a postural icon P1 representing the postural state of the cattle individual. The postural icon P1, which is an icon representing that the cattle individual has been in the non-prostrate state, may be displayed, for example, in a manner that the head of the cattle individual is vertically oriented.

In addition, when the user terminal 4 manages the plurality of sensor devices 1, as shown in FIG. 12, the sensor-information presentation screen 441 may include the plurality of sensor information fields S. This enables the user to grasp the sensor information items of the plurality of sensor devices 1 at a glance.

A of FIG. 13 shows a screen 442 including the first-astasia notification information item.

In an example shown in A of FIG. 13, the first-astasia notification information item is distributed as the push notification, and displayed in a dialog box D1.

The dialog box D1 displays, as the first-astasia notification information item, an alert message indicating that the inference that the first astasia has developed is made, and the sensor information item of the sensor device 1 (identifier of the sensor device 1, attachment start date of the sensor device 1, the breeding site of the livestock animal wearing the sensor device 1 in the stockbreeding facility, and the identification number of the livestock animal wearing the sensor device 1), which relates to the notification information item. Examples of the alert message include a phrase such as “Stay Alert for Astasia” or “Alert,” a color of the dialog box (such as yellow being an alert color), a postural icon P2 representing that the head of the cattle individual is horizontally oriented, a date when the inference that the first astasia has developed is made, and the like.

This enables the user who receives the push notification to recognize that the livestock animal relating to this notification has been in the prostrate state for the first duration or longer, and is likely to develop the astasia.

In addition, the dialog box D1 includes an OK button B1. By an instruction issued by an input operation such as a tap by the user to the OK button B1, the display unit 44 can display a sensor-information presentation screen 443 shown in B of FIG. 13.

The sensor-information presentation screen 443 shown in B of FIG. 13 includes the sensor information fields S similar to those on the sensor-information presentation screen 441 shown in FIG. 12, and a sensor information field S1 about the sensor device 1 from which the first-astasia notification data item has been transmitted. The sensor information field S1 displays not only the information items included in the sensor information field S but also the above-mentioned alert message. This enables the user to check detailed information items about the livestock animal that is likely to develop the astasia, and its sensor device 1.

A of FIG. 14 shows a screen 444 including the second-astasia notification information item.

In an example shown in A of FIG. 14, similar to the first-astasia notification information item, the second-astasia notification information item is distributed as the push notification, and displayed in a dialog box D2. The dialog box D2 displays, as the second-astasia notification information item, a warning message indicating that the inference that the second astasia has developed is made, and the sensor information item of the sensor device 1. Examples of the warning message include a phrase such as “Warn of Astasia” or “Warning,” a color of the dialog box (such as red being a warning color), the postural icon P2 representing that the head of the cattle individual is horizontally oriented, a date when the inference that the second astasia has developed is made, and the like.

This enables the user who receives the push notification to recognize that the livestock animal relating to this notification has been in the prostrate state for the second duration or longer, and is highly likely to have already developed the astasia.

In addition, the dialog box D2 includes an OK button B2. By an instruction issued by the input operation such as the tap by the user to the OK button B2, the display unit 44 can display a sensor-information presentation screen 445 shown in B of FIG. 14.

The sensor-information presentation screen 445 shown in B of FIG. 14 includes the sensor information fields S similar to those on the sensor-information presentation screen 441 shown in FIG. 12, and a sensor information field S2 about the sensor device 1 from which the second-astasia notification data item has been transmitted. The sensor information field S2 displays not only the information items included in the sensor information field S but also the above-mentioned warning message. This enables the user to check detailed information items about the livestock animal that is highly likely to have developed the astasia, and its sensor device 1.

Note that, also when the user terminal 4 receives the astasia-clearance notification information item from the server 3, the user terminal 4 may display a push notification similar to those of the astasia-notification information items. Alternatively, the user terminal 4 need not necessarily display the push notification, and may display that the astasia has been cleared on the sensor-information presentation screen.

As described hereinabove, the livestock management system 100 according to this embodiment enables the user to be notified that the livestock animal is likely to have developed the astasia. This enables the user also to provide prompt treatment to the livestock animal that has developed the astasia.

For example, livestock animals such as the fed cattle are liable to develop the astasia once being prostrated. Further, when such livestock animals are in the prostrate state for several hours, these livestock animals may end up dead due to ruminal abnormal fermentation called bloat. In other words, the development of the astasia of the livestock animals may cause serious damage to livestock farmers.

As a countermeasure, hitherto, the stockbreeders have monitored the livestock animals, stimulated a livestock animal in the prostrate state so as to check whether or not this livestock animal has developed the astasia (that is, whether or not to be able to stand up by itself), and provided treatment such as assistance of pulling up the livestock animal when the livestock animal is unable to stand up by itself.

By introduction of the livestock management system 100 according to this embodiment enables, the users being the stockbreeders can accurately grasp that the livestock animal has developed the astasia even without working on frequent monitoring of the livestock animal for checking whether or not the livestock animal is able to stand up by itself. With this, damage to the users due to the astasia of the livestock animals can be prevented, and effort in the monitoring work and the like can be significantly reduced.

In addition, with regard to the livestock animals, the frequency of being stimulated for checking whether or not the astasia has developed can be reduced. Thus, stress can be alleviated, and qualities such as a meat quality can be enhanced.

### Modifications of this Embodiment

Now, modifications of this embodiment are described. Note that, components that are similar to those described above in this embodiment are denoted by the same reference symbols to omit description thereof.

(Modification 1: Modification of Attachment That Enables Attachment of Sensor Device)

The attachment 17 is not limited to the above-described one including the cord 171.

As illustrated in FIG. 15, the attachment 17 may include, instead of the cord 171, a belt 172 and a length adjustment portion 173 including, for example, an adjuster capable of adjusting a length of the belt 172. Also with this, the adjustment to the appropriate length in accordance with the size of the head of the livestock animal A can be performed, and stress to the livestock animal A can be alleviated.

(Modification 2: Modification of Attachment Position of Sensor Device)

The casing 10 of the sensor device 1 may be attached to a part other than the submental part of the head of the livestock animal A.

As illustrated in FIG. 16, the casing 10 may be configured to be capable of being attached to the occipital part of the livestock animal A. Also with this, the postural state of the livestock animal can be accurately determined. Note that, instead of the belt 172 of the attachment 17 in the example illustrated in FIG. 16, the cord 171 illustrated in FIG. 3 may be used.

Alternatively, although not shown, the casing 10 may be attached to the supranasal part of the livestock animal A, or may be attached, for example, to the forehead (sincipital part) of the same. Still alternatively, the casing 10 may be configured to be capable of being attached to parts out of the head of the livestock animal A. As examples of these parts, there may be mentioned, for example, the trunk (back, abdomen, chest, buttocks, or the like) and legs (forelimbs and hindlimbs).

(Modification 3: Modification of Making Inference as to Whether or Not Astasia Notification State has Developed in Step with Numbers Other than Two)

The state inference unit 103 (control unit 13) need not necessarily be configured to infer whether or not the astasia has developed in the two steps, and may be configured to infer once that the astasia has developed when the prostrate state has continued for a predetermined duration or longer.

Alternatively, the state inference unit 103 may infer whether or not the astasia has developed in three or more steps.

(Modification 4: Modification of Transmission of Astasia-Clearance Notification Data Item)

In the above-described operation examples, the control unit 13 is configured not to transmit the astasia-clearance notification data item after inferring that the second astasia has developed because the control unit 13 ends the inference procedure after inferring that the second astasia has developed.

The control unit 13 need not necessarily be configured in this way, and may transmit the astasia-clearance notification data item after inferring that the second astasia has developed. In this case, the control unit 13 may continue the inference procedure by continuing the timekeeping with the timer 133 until determining that the livestock animal has been in the non-prostrate state.

### Second Embodiment

In the above-described first embodiment, the sensor device transmits the astasia-notification data item when the inference that the astasia has developed is made. In addition to this, the sensor device may transmit, periodically to the server, a data item about the postural state of the livestock animal, which does not include the astasia-notification data item.

[Summary of Another Livestock Management System]

FIG. 17 is a schematic view illustrating a schematic configuration of a livestock management system 100A according to this embodiment.

The livestock management system 100A includes a plurality of sensor devices 1A, the relay device 2, a server 3A, and the user terminal 4. In FIG. 17, illustration of the livestock animal is omitted.

Note that, hereinbelow, components that are similar to those in the above-described first embodiment are denoted by the same reference symbols to omit description thereof.

As in the first embodiment, the sensor devices 1A each execute the astasia inference procedure for the livestock animal, and transmit the astasia-notification data item. In addition, the sensor devices 1A are each capable of executing a postural-data transmission procedure of transmitting, periodically to the server 3, the postural data item that does not include the astasia-notification data item.

As in the first embodiment, the server 3A generates and transmits the astasia-notification information item including the astasia-notification data item to the user terminal 4.

In addition, in this embodiment, the server 3A is capable of not only generating the astasia-notification information item of each of the livestock animals, but also executing a postural-data accumulation procedure of storing the postural data item of each of the livestock animals.

[Functional Configuration of Another Livestock Management System]

FIG. 18 is a block diagram showing a functional configuration of the livestock management system 100A according to this embodiment.

In this embodiment, the sensor devices 1A each include the detection unit 101, a postural-state determination unit 102A, a state inference unit 103A, a transmission unit 104A, and the casing 10. Although not shown in FIG. 18, since the livestock management system 100A according to this embodiment includes the plurality of sensor devices 1A, the livestock management system 100A may include a plurality of postural-state determination units 102A, a plurality of state inference units 103A, a plurality of transmission units 104A, and a plurality of casings 10.

As in the first embodiment, the relay device 2 includes the relay unit 105.

In this embodiment, the server 3A includes the notification-information generation unit 106 and a postural-data storage unit 108.

In this embodiment, the user terminal 4 includes the notification unit 107 similar to that in the first embodiment.

Note that, the respective hardware configurations of the devices are the same as the configurations shown in FIG. 4, and hence description thereof is omitted.

The postural-state determination unit 102A is capable of determining in which of the prostrate posture and the non-prostrate posture corresponding one of the plurality of livestock animals has been on the basis of the output values from the acceleration sensor 121.

In addition, the postural-state determination unit 102A is capable of causing the memory 132 to store results of the determinations together with dates and time points of the determinations after determining in which of the prostrate state and the non-prostrate state the livestock animal has been on the basis of the output values from the acceleration sensor 121. Specifically, the postural-state determination unit 102A is capable of causing the memory 132 to store information items about the postural states in which the livestock animal has been determined to have been, and information items about the dates and the time points of the determinations in correspondence with each other.

The state inference unit 103A infers whether or not corresponding one of the plurality of livestock animals has developed the astasia on the basis of the duration of the prostrate state.

As in the first embodiment, when the inference that the livestock animal has developed the astasia is made, the transmission unit 104A transmits, to the server 3A, the astasia-notification data item indicating that the inference that the astasia has developed is made.

In addition, in this embodiment, the transmission unit 104A transmits, periodically to the server 3, the data item that includes a postural-state information item about in which of the prostrate state and the non-prostrate state the livestock animal has been, which is determined by the postural-state determination unit 102A, and that does not include the astasia-notification data item. The transmission unit 104A can correspond, for example, to the control unit 13 and the communication unit 14.

In this embodiment, the postural data item may include the postural-state information item and the identification information item of the livestock animal. The postural-state information item includes at least an information item about the postural state of the livestock animal at the time of transmission of the postural data item. In addition, when the postural-state determination unit 102A has determined that the livestock animal has been in a different postural state between a previous transmission and a current transmission, the postural-state information item may include information items about postural states before and after the determination, and a determination date and a determination time point of the determination.

The transmission unit 104A may transmit the postural data item at a predetermined interval to the server 3. This predetermined interval, which is not particularly limited, may be set, for example, to from approximately several minutes to 72 hours.

In addition, the transmission unit 104A is capable of transmitting the astasia-notification data item with priority over the postural data item. This enables the user to be notified of the astasia-notification data item, which is desired to be promptly notified of, to the user can be performed with the high priority.

Specifically, in the case where the transmission process to the server 3 fails, the transmission unit 104A may execute the retry process. In this embodiment, the transmission unit 104A is configured to be capable of executing the retry processes the larger number of times at the time of the process of transmitting the astasia-notification data item than that at the time of the process of transmitting the postural data item. More specifically, the transmission unit 104A is set to unlimitedly execute the retry process until the transmission process is successfully executed at the time of the process of transmitting the astasia-notification data item, and set to execute the retry process a predetermined number of times (for example, several times) at the time of the process of transmitting the postural data item. With this, the astasia-notification data item can be further reliably notified of.

The postural-data storage unit 108 stores the postural data items of the plurality of livestock animals. The postural-data storage unit 108 can correspond, for example, to the control unit 31 and the storage unit 32 of the server 3A. As described above, the postural data items each include the postural-state information item about in which of the prostrate state and the non-prostrate state corresponding one of the plurality of livestock animals has been, and none of the postural data items includes the astasia-notification data item. The postural-data storage unit 108 is capable of accumulating the postural data items transmitted respectively from the livestock animals.

[Operation Example of Another Livestock Management System]

FIG. 19 is a flowchart showing an operation example of the postural-data accumulation procedure in the livestock management system.

In FIG. 19, processes of S401 and S402 are executed by the sensor device 1A, processes of S403 and S404 are executed by the relay device 2, and processes of S405 to S406 are executed by the server 3A.

Note that, the astasia notification procedure is similar to that in the operation example of the first embodiment, which is described with reference to FIG. 9 and FIG. 10. Thus, description thereof is omitted.

First, the control unit 13 of the sensor device 1A determines whether or not a time point of transmitting the postural data item has been reached (S401). When the control unit 13 determines that the time point of the transmission has been reached (YES in S401), the sensor device 1A transmits the postural data item (S402). In this operation example, the postural data item includes the postural-state information item and the identification information item of the livestock animal, which are stored in the memory 132. When the postural-state determination unit 102A has determined that the livestock animal has been in a different postural state between a previous transmission and a current transmission, the postural-state information item includes the information items about the postural states before and after the determination, and the determination date and the determination time point of the determination.

Note that, as described above, the sensor device 1 may execute the retry process in the case where the transmission process fails.

Then, the relay device 2 receives the postural data item (S403), and transmits this postural data item to the server 3A (S404).

Next, the server 3A receives the postural data item (S405), and the storage unit 32A of the server 3A stores this postural data item (S406).

In this way, the server 3A is capable of accumulating the respective postural-data items of the livestock animals. The accumulated postural-data items can be utilized as follows.

For example, the server 3 may transmit the postural data items to the user terminal 4 at predetermined timings. With this, the user terminal 4 can reflect the information items of the transmitted postural-data items, for example, to the postural icons on the sensor-information presentation screen 441 shown in FIG. 12. In addition, the user terminal 4 may enable a check on a temporal transition of the postural data item of corresponding one of the livestock animals by the operation such as the tap to the sensor information field S.

Alternatively, the server 3 may analyze, for example, correlations between the postural states and the meat quality on the basis of the accumulated postural-data items.

### Third Embodiment

In the second embodiment, the capability of the server in accumulating the postural data items is described. In this embodiment, as another example of utilizing the accumulated postural-data items, an example in which the server analyzes respective risks that the livestock animals develop the astasia is described.

[Functional Configuration of Still Another Livestock Management System]

FIG. 20 is a diagram showing a functional configuration of a livestock management system 100B according to this embodiment.

The livestock management system 100B includes, similar to the livestock management system 100A, the plurality of sensor devices 1A, the relay device 2, a server 3B, and a user terminal 4B (refer to FIG. 17).

Note that, hereinbelow, components that are similar to those in each of the above-described embodiments are denoted by the same reference symbols to omit description thereof.

As in the second embodiment, the sensor devices 1A each include the detection unit 101, the postural-state determination unit 102A, the state inference unit 103A, the transmission unit 104A, and the casing 10.

As in the first embodiment, the relay device 2 includes the relay unit 105.

The server 3B includes not only the notification-information generation unit 106 and the postural-data storage unit 108 that are similar to those in the second embodiment, but also a notification-information storage unit 109 and an analysis unit 110.

The user terminal 4B includes not only the notification unit 107 similar to that in the first embodiment, but also an analysis-result presentation unit 111B.

The respective hardware configurations of the devices are the same as the configurations shown in FIG. 4, and hence description thereof is omitted.

The notification-information storage unit 109 is capable of storing the astasia-notification information items of the plurality of livestock animals, which respectively include the astasia-notification data items each indicating that the inference that corresponding one of the livestock animals has developed the astasia is made. The notification-information storage unit 109 can correspond, for example, to the control unit 31 and the storage unit 32 of the server 3B.

The analysis unit 110 is capable of analyzing the respective risks that the plurality of livestock animals develops the astasia on the basis of the respective astasia-notification information items of the livestock animals, and on the basis of the postural data items of the plurality of livestock animals. The analysis unit 110 can correspond, for example, to the control unit 31 of the server 3B.

Specifically, the analysis unit 110 is capable of analyzing temporal patterns of the postural states of ones of the livestock animal, to which the inferences that the astasia has developed are made, and thereby capable of analyzing the respective risks that these livestock animals develop the astasia on the basis of results of the analyses and the respective temporal transitions of the postural states of the livestock animals. The analysis unit 110 is capable of performing machine learning that uses, as supervised cases, the temporal patterns of the postural states of the ones of the livestock animal, to which the inferences that the astasia has developed are made, and thereby capable of generating an algorithm the algorithm that derives the risks that the livestock animals develop the astasia from the respective temporal transitions of the postural states of these livestock animals.

Examples of the patterns of the postural states include patterns of the number of occurrences of the prostrate state, and patterns of the durations of the prostrate state within a predetermined time period.

As examples of output of the results of the analyses, for example, identification information items and sensor information items of the high-risk ones of the livestock animals may be output, or results of evaluations of a risk of the one or the plurality of livestock animals may be output.

The analysis-result presentation unit 111B presents the results of the analyses by the analysis unit 110 to the user. The analysis-result presentation unit 111B can correspond, for example, to the control unit 41 and the display unit 44 of the user terminal 4.

The analysis-result presentation unit 111B is capable of presenting the analysis results generated by the server 3B to the user, for example, by causing the display unit 44 to display these analysis results. Alternatively, the analysis-result presentation unit 111B may present the analysis results by voice via the speaker or the like, or may present the analysis results in the form of vibration or the like. Still alternatively, the results may be presented by combining these plurality of presentation methods with each other.

The analysis-result presentation unit 111B may, for example, request the server 3B to transmit the analysis results, and present the received analysis results. Alternatively, the analysis results that the analysis-result presentation unit 111B present may be transmitted at predetermined timings from the server 3B. Examples of the predetermined timings include a timing of activating the livestock management application in the user terminal 4, predetermined intervals or time points, or the like. In addition, when an evaluation that a certain livestock animal is highly likely to develop the astasia is made, for example, by a periodical analysis process by the server 3B (analysis unit 110), the analysis-result presentation unit 111B may present a result of this analysis, which is transmitted from the server 3B.

The analysis-result presentation unit 111B may present, as the analysis results, for example, a list of livestock animals that are evaluated to be highly likely to develop the astasia, and data items about risks that livestock animals specified at the time of requesting the transmission develop the astasia.

As described above, according to this embodiment, the livestock animals that are highly likely to develop the astasia can be grasped. This enables the user to provide treatment such as monitoring intensively on the livestock animals that are highly likely to develop the astasia. As a result, the damage to be caused by the astasia of the livestock animals can be further reliably prevented.

### Fourth Embodiment

In the above-described first embodiment, the sensor device 1 infers whether or not the astasia has developed on the basis of the durations of the prostrate state. Meanwhile, the livestock animals such as cattle are known to struggle to try to stand up by themselves in a case of developing the astasia. As a countermeasure, in this embodiment, the sensor device 1 executes not only the above-described astasia inference procedure but also detect a predetermined vibration after determining that the livestock animal has been in the prostrate state. With this, the sensor device 1 infers whether or not the livestock animal has been in a self-standing-attempt state that accompanies the astasia of the livestock animal.

[Configuration of Yet Another Livestock Management System]

FIG. 21 is a diagram showing hardware configurations in a livestock management system 100C according to this embodiment.

The livestock management system 100C includes a sensor device 1C, the relay device 2, a server 3C, and the user terminal 4.

Note that, hereinbelow, components that are similar to those in each of the above-described embodiments are denoted by the same reference symbols to omit description thereof.

The sensor device 1C includes the power supply unit 11, a sensor unit 12C, a control unit 13C, and the communication unit 14.

The sensor unit 12C includes the acceleration sensor 121, the first comparator 122, a first counter 123, the second comparator 124, a third comparator 125, a second counter 126, and a fourth comparator 127.

The first comparator, the first counter 123, and the second comparator, which correspond respectively to the first comparator, the counter 123, and the second comparator in the first embodiment, execute the detection-signal output procedure.

The third comparator 125, the second counter 126, and the fourth comparator 127 execute a procedure of outputting a vibration detection signal to be used in a self-standing-attempt-state inference procedure according to this embodiment.

The third comparator 125 and the fourth comparator 127, each of which is a comparator circuit that compares an input value and a threshold to each other, perform output when the input value is larger than the threshold. The second counter 126 is a counter circuit that counts the output from the third comparator 125 in a predetermined sampling period.

The control unit 13C includes the processor 131, the memory 132, a first timer 133, a second timer 134, and a third counter 135.

The first timer 133 corresponds to the timer 133 in the first embodiment.

The second timer 134 and the third counter 135 are used for the self-standing-attempt-state inference procedure according to this embodiment.

The second timer 134 is a timer that measures time periods at timings different from those of the first timer 133. When to start and stop the timekeeping is controlled by the processor 131.

The third counter 135 is a counter circuit that counts the number of times of outputting the vibration detection signal.

[Functional Configuration of Yet Another Livestock Management System]

FIG. 22 is a block diagram showing a functional configuration of the livestock management system 100C according to this embodiment.

In this embodiment, the sensor device 1C includes not only the detection unit 101, the postural-state determination unit 102, a state inference unit 103C, a transmission unit 104C, and the casing 10, but also a vibration detection unit 112C.

As in the first embodiment, the relay device 2 includes the relay unit 105.

The server 3 includes the notification-information generation unit 106 similar to that in the first embodiment.

The user terminal 4 includes the notification unit 107 similar to that in the first embodiment.

As in the first embodiment, the detection unit 101 outputs the detection signal when the output values from the acceleration sensor 121 include output values that are each larger than a first preset-output value and that continue to be detected. The first preset-output value corresponds to the preset output value in the first embodiment. The detection unit 101 can correspond to the acceleration sensor 121, the first comparator 122, the first counter 123, and the second comparator 124 of the sensor unit 12C.

The vibration detection unit 112C outputs the vibration detection signal when the output values from the acceleration sensor 121 include output values that are each larger than a second preset-output value and that continue to be detected. The vibration detection unit 112C can correspond to the acceleration sensor 121, the third comparator 125, the second counter 126, and the fourth comparator 127 of the sensor unit 12C.

The vibration detection unit 112C is capable of outputting the vibration detection signal, for example, when the output values from the acceleration sensor 121 include output values that are each larger than the second preset-output value and that are detected for a predetermined vibration-determination time period or longer.

The second preset-output value is not particularly limited as long as output values corresponding to dynamic motions of the livestock animal can be detected. For example, this second preset-output value may be set to a value larger than the first preset-output value.

The vibration-determination time period is not particularly limited as long as whether or not the livestock animal has been in the struggling state can be determined. For example, this vibration-determination time period may be set to several seconds or more and less than several minutes. The vibration-determination time period may be equal to or unequal to the state-determination time period.

The vibration detection signal may be a signal that requests an interruption procedure into the control unit 13C, which may include, for example, a flag indicating the request for the interruption procedure.

The vibration detection unit 112C is capable of outputting the vibration detection signal by processes as follows.

First, when the output values from the acceleration sensor 121 are each larger than the second preset-output value, the third comparator 125 outputs these output values as a signal to the second counter 126.

The second counter 126 counts the output signals from the third comparator 125 in the predetermined sampling period.

When the count value from the second counter 126 becomes larger than a second preset-count value, the fourth comparator 127 outputs a processing result as the vibration detection signal to the control unit 13C. The second preset-count value is, for example, a value to be calculated by dividing the vibration-determination time period by the sampling period of the second counter 126.

In this way, the fourth comparator 127 can output the vibration detection signal when a time period of the output from the third comparator 125 is longer than the vibration-determination time period.

As in the first embodiment, the postural-state determination unit 102 determines in which of the prostrate state and the non-prostrate state the livestock animal has been on the basis of the output values from the acceleration sensor 121.

Similar to the state inference unit 103, the state inference unit 103C infers whether or not the livestock animal has developed the astasia on the basis of the durations of the prostrate state.

The state inference unit 103C is also capable of inferring that the livestock animal has been in the self-standing-attempt state that accompanies the astasia of the livestock animal when the output values each larger than the preset second-output value are detected from the acceleration sensor 121 a predetermined number of times or more within a predetermined time period after the postural-state determination unit 102 has determined that the livestock animal has been in the prostrate state.

Hereinbelow, the predetermined time period is defined as a preset monitoring-time period, and the predetermined number of times is defined as a preset number of times of vibrations.

The state inference unit 103C may infer not only whether or not the livestock animal has developed the astasia on the basis of the durations of the prostrate state, but also whether or not the livestock animal has been in the self-standing-attempt state that accompanies the astasia.

Alternatively, the state inference unit 103C may use the self-standing-attempt-state inference procedure as the astasia inference procedure. In other words, the state inference unit 103C may infer that the livestock animal has developed the astasia when the prostrate state satisfies the conditions of the durations, and at the same time, when the inference that the livestock animal has been in the self-standing-attempt state is made.

In this case, the state inference unit 103C may use the self-standing-attempt-state inference procedure as any one of the inference as to whether or not the first astasia has developed and the inference as to whether or not the second astasia has developed. For example, the state inference unit 103C may use the self-standing-attempt-state inference procedure as the processes of inferring whether or not the second astasia has developed.

When the inference that the livestock animal has developed the astasia is made, the transmission unit 104C transmits, to the server 3, the astasia-notification data item indicating that the inference that the astasia has developed is made. In this embodiment, the transmission unit 104 is also capable of transmitting, to the server 3, a notification data item of the self-standing-attempt state when the inference that the livestock animal has been in the self-standing-attempt state is made.

[Operation Examples of Sensor Device]

FIG. 23 and FIG. 24 are flowcharts showing operation examples of the sensor device 1C. With reference to these drawings, an operation example in which the sensor device 1C executes a vibration-detection-signal output procedure of generating the vibration detection signal, and an operation example in which the sensor device 1C executes the astasia inference procedure of inferring whether or not the livestock animal has developed the astasia on the basis of the vibration detection signal are described.

(Vibration-Detection-Signal Output Procedure)

FIG. 23 is a flowchart showing the operation example of the vibration-detection-signal output procedure by the sensor unit 12C (vibration detection unit 112C).

First, the third comparator 125 compares each of the output values from the acceleration sensor 121 and the second preset-output value for the vibration detection to each other (S501). When all these output values are larger than the preset second-output value (YES in S501), the third comparator 125 outputs an output signal.

In this operation example, the output values from the acceleration sensor 121 may be the output values corresponding to any of the detection axes, and the second preset-output value may be the value larger than the first preset-output value.

The second counter 126 performs count-up on the basis of the output signals from the third comparator 125 (S502). The second counter 126 counts up the output signals from the third comparator 125 at the predetermined sampling interval. In other words, the second counter 126 continues the count-up while the third comparator 125 successively outputs the output signals.

Meanwhile, when the output value from the acceleration sensor 121 becomes smaller than the second preset-output value (NO in S501), the third comparator 125 stops outputting the output signal, and the second counter 126 does not receive the output signal. In this case, the second counter 126 clears the count value (S503), and the procedure is ended.

The fourth comparator 127 compares the count value from the second counter 126 and the preset count value to each other (S504). When the count value becomes larger than the preset count value (YES in S504), the fourth comparator 127 outputs the vibration detection signal (S505). The vibration detection signal, which includes the interruption flag, is transmitted to the control unit 13C. The preset count value is, for example, a value calculated by dividing the vibration-determination time period by the sampling period of the second counter 126. The vibration-determination time period may be set, for example, to from several seconds to several minutes.

By the above-described procedure, when the output values from the acceleration sensor 121 include the output values that are each larger than the second preset-output value for the vibration detection and that are detected for the predetermined vibration-determination time period or longer, the sensor unit 12C can output the vibration detection signal. In the self-standing-attempt-state inference procedure, the vibration detection signal is treated as a signal indicating detection of a single vibration.

(Self-Standing-Attempt-State Inference Procedure)

FIG. 24 is a flowchart showing the operation example of the self-standing-attempt-state inference procedure in which the vibration detection signal from the control unit 13C (postural-state determination unit 102, state inference unit 103C, and transmission unit 104) is used.

In the self-standing-attempt-state inference procedure of this operation example, not only the inferences based on the durations of the prostrate state as to whether or not the first astasia has developed and whether or not the second astasia has developed are made, but also the inference based on the vibration detection signal as to whether or not the livestock animal has been in the self-standing-attempt state that accompanies the astasia is made.

First, the control unit 13C determines whether or not the determination that the livestock animal has been in the prostrate state has been made (S601). Only when the determination that the livestock animal has been in the prostrate state has been made (YES in S601), the procedure proceeds to S602.

When the control unit 13C receives the vibration detection signal from the sensor unit 12C (YES in S602), the control unit 13C determines whether or not the second timer 134 has been stopped (S603). When the second timer 134 has been stopped (YES in S603), the control unit 13C activates the second timer 134 (S604). The second timer 134 measures the monitoring time period during which the reception of the vibration detection signal is monitored.

The self-standing-attempt-state inference procedure of this operation example, in which the vibration detection signal is used, is defined as an interruption procedure based on the vibration detection signal. Thus, in S602, the control unit 13C need not always monitor the reception of the vibration detection signal, and only needs to execute the process of S603 and subsequent processes when the interruption request by the vibration detection signal is received from the sensor unit 12C.

Then, immediately after the control unit 13C activates the second timer 134 (S604), the control unit 13C clears a count value in the third counter 135 (S605), and causes the third counter 135 to perform count-up on the basis of the reception of the vibration detection signal (S606).

The third counter 135 is a counter that counts the number of times of the reception of the vibration detection signal (number of times of vibrations) within the preset monitoring-time period.

When the count value remains equal to or smaller than the preset number of times of vibrations even after the count-up by the third counter 135 (NO in S607), the control unit 13C interrupts the interruption procedure, and causes the procedure to return to S602.

When the control unit 13C receives the vibration detection signal again (YES in S602), since the second timer has been performing timekeeping (NO in S603), the control unit 13C determines whether or not the time period measured by the second timer is shorter than the preset monitoring-time period (S608). When this measured time period is shorter than the preset monitoring-time period (YES in S608), the third counter 135 continues the count-up (S606).

Then, when the count value in the third counter 135 becomes larger than the preset number of times of vibrations (YES in S607), the control unit 13C determines that a larger number of times of vibrations than the preset number of times of vibrations have been detected within the preset monitoring-time period, and infers that the livestock animal has been in the self-standing-attempt state (S609). With this, the control unit 13C clears the count value in the third counter 135 (S610), and stops the second timer 134 (S611).

Lastly, the control unit 13C generates a self-standing-attempt notification data item indicating that the inference that the livestock animal has been in the self-standing-attempt state has been made, causes the communication unit 14C to execute a transmission process (S612), and then ends the procedure.

The self-standing-attempt notification data item includes, for example, a flag indicating that the inference that the livestock animal has been in the self-standing-attempt state has been made, an information item about a date and a time point when the inference that the livestock animal has been in the self-standing-attempt state has been made, and the identification information item of the livestock animal.

Note that, the control unit 13C is capable of executing the self-standing-attempt-state inference procedure of this operation example, for example, after at least one of the inference that the first astasia has developed or the inference that the second astasia has developed has been made. In this case, the communication unit 14C is capable of collectively transmitting at least one of the generated first-astasia notification data item or the generated second-astasia notification data item, and the self-standing-attempt notification data item.

Alternatively, the control unit 13C may execute only the self-standing-attempt-state inference procedure of this operation example when none of the inference that the first astasia has developed and the inference that the second astasia has developed has been made. In this case, the communication unit 14C may transmit only the self-standing-attempt notification data item.

Further, the notification-information generation unit 106 of the server 3 is capable of generating, after receiving the self-standing-attempt notification data item, a self-standing-attempt notification information item including the self-standing-attempt notification data item indicating that the inference that the livestock animal has been in the self-standing-attempt state has been made. Still further, the notification unit 107 of the user terminal 4 is capable of notifying the user of the self-standing-attempt notification information item. The self-standing-attempt notification information item includes at least an information item about the self-standing-attempt notification data item, and may further include, for example, a sensor information item of the sensor device 1C, which relates to the notification data item.

As described above, the sensor device 1C according to this embodiment regards the livestock animal as being struggling to try to stand up by itself when frequently detecting the dynamic motions of the livestock animal. With this, the inference that the livestock animal has been in the self-standing-attempt state can be made. In this way, the sensor device 1C is capable of further accurately inferring that the livestock animal has been in the state relating to the astasia.

### Modification of Fourth Embodiment

As a modification of this embodiment, when the prostrate state has continued for the durations or longer, and at the same time, when the inference that the livestock animal has been in the self-standing-attempt state is made, the inference that the astasia has developed may be made.

For example, when the control unit 13C (state inference unit 103C) detects that the prostrate state has continued for the second duration or longer, and detects the larger number of times of vibrations than the preset number of times of vibrations within the preset monitoring-time period, the control unit 13C may infer that the second astasia has developed. Specifically, the control unit 13C may execute the interruption processes of from S602 to S608 in FIG. 24 between S208 and S209 in FIG. 10. Then, when the time period measured by the first timer is equal to or longer than the second state-inference time period (YES in S209), and at the same time, when the count value in the third counter 135 becomes larger than the preset number of times of vibrations (YES in S607), the control unit 13C may infer that the second astasia has developed.

In addition, also in making the inference as to whether or not the first astasia has developed, the control unit 13C (state inference unit 103C) may use not only the conditions of the durations but also the conditions of the vibration detection. In this case, the conditions such as the preset number of times of vibrations and the preset monitoring-time period may be applied differently from each other in accordance with the first astasia and the second astasia.

### Other Embodiments

Further, the present technology may also employ embodiments as follows.

FIG. 25 is a block diagram showing a functional configuration of a livestock management system 100D according to another embodiment of the present technology.

For example, as in a sensor device 1D shown in FIG. 25, the detection unit need not necessarily be provided, and a postural-state determination unit 102D may determine in which of the prostrate state and the non-prostrate state the livestock animal has been on the basis of the output values from the acceleration sensor 121. Specifically, the sensor device 1D may include the postural-state determination unit 102D, the state inference unit 103, and the transmission unit 104, the server 3 may include the notification-information generation unit 106, and the user terminal 4 may include the notification unit 107. This enables the control unit 13 of the sensor device 1D to directly monitor the output values from the acceleration sensor 121, and to determine that the livestock animal has been in the prostrate state when the output values include the output values that are each larger than the preset output value and that are detected for the state-determination time period or longer.

FIG. 26 is a block diagram showing a functional configuration of a livestock management system 100E according to still another embodiment of the present technology.

In the livestock management system 100E, a sensor device 1E is capable of executing only the detection-signal output procedure, and a server 3E is capable of executing the astasia inference procedure and the astasia notification procedure.

Specifically, the sensor device 1E includes only the detection unit 101E that outputs the detection signal, and the server 3E includes a postural-state determination unit 102E that determines in which of the prostrate state and the non-prostrate state the livestock animal has been on the basis of the detection signal, a state inference unit 103E, and a notification-information generation unit 106E. The postural-state determination unit 102E and the state inference unit 103E can correspond to the control unit 31 of the server 3E.

Also with this, the astasia inference procedure and the astasia notification procedure similar to those in the first embodiment can be executed.

Alternatively, as shown in FIG. 27, a livestock management system 100F may include a sensor device 1F that does not include the detection unit as that in the livestock management system 100D does not.

Specifically, the sensor device 1F includes a transmission unit 104F that transmits, to a server 3F, the output values from the acceleration sensor 121. The server 3F includes a postural-state determination unit 102F that determines in which of the prostrate state and the non-prostrate state the livestock animal has been on the basis of the output values, the state inference unit 103E, and the notification-information generation unit 106E.

Also with this, the astasia inference procedure and the astasia notification procedure similar to those in the first embodiment can be executed.

In addition, the livestock management systems may include a plurality of user terminals. With this, the notification information items can be transmitted to a plurality of users who works at a stockbreeding facility.

The livestock management systems may include a plurality of relay devices. This enables, even when the livestock animals wearing the sensor devices are present in a relatively large region by grazing or the like, the relay devices to further reliably perform communication between the server and the sensor devices. In this case, the notification data items and the postural data items that the relay devices transmit to the server may include, for example, device information items indicating from which of the relay devices these data items are transmitted.

Alternatively, as shown in FIG. 28, the livestock management system may be configured not to include the relay device such that the sensor device is connected to the server by being directly connected to the network. In this case, as in a livestock management system 100G shown in FIG. 28, the relay unit 105 need not necessarily be provided.

Still alternatively, when the sensor device can be directly connected to the network, the livestock management system need not necessarily include the server such that the sensor device transmits the notification data items directly to the user terminal. In this case, the sensor device may include the detection unit, the postural-state determination unit, the state inference unit, and the transmission unit, and the user terminal may include the notification-information generation unit and the notification unit.

As a matter of course, the present technology is not limited only to the embodiments described hereinabove of the present technology, and various changes may be made without departing from the gist of the present technology. For example, the embodiments of the present technology may be combined with each other.

Note that, the present technology may also employ configurations as follows.
(1) A livestock sensor device, including:

a postural-state determination unit that determines in which of a prostrate state and a non-prostrate state a livestock animal has been on a basis of output values from an acceleration sensor;

a state inference unit that infers whether or not the livestock animal has developed astasia on a basis of a duration of the prostrate state;

a transmission unit that transmits, to a server, an astasia-notification data item indicating that an inference that the livestock animal has developed the astasia is made when the inference that the livestock animal has developed the astasia is made; and

a casing that is configured to be
capable of housing
   the acceleration sensor,
   the postural-state determination unit,
   the state inference unit, and
   the transmission unit, and
capable of being attached to a head of the livestock animal.
(2) The livestock sensor device according to (1), further including

a detection unit that outputs a detection signal when the output values from the acceleration sensor include output values that are each larger than a preset output value and that continue to be detected, in which

the postural-state determination unit determines in which of the prostrate state and the non-prostrate state the livestock animal has been on a basis of the detection signal.
(3) The livestock sensor device according to (1) or (2), in which

the acceleration sensor has a plurality of detection axes,

the plurality of detection axes includes
a prostration detection axis that enables detection of a highest acceleration in a gravity direction from among the plurality of detection axes in the prostrate state of the livestock animal,
a non-prostration detection axis that enables detection of the highest acceleration in the gravity direction from among the plurality of detection axes in the non-prostrate state of the livestock animal,
the postural-state determination unit
   determines that the livestock animal has been in the prostrate state on a basis of ones of the output values, the ones corresponding to the prostration detection axis, and
   determines that the livestock animal has been in the non-prostrate state on a basis of other ones of the output values, the other ones corresponding to the non-prostration detection axis.
(4) The livestock sensor device according to (3), in which

the postural-state determination unit includes
a prostrate-state determination mode in which a determination that the livestock animal has been in the prostrate state can be made on the basis of the ones of the output values, the ones corresponding to the prostrate-posture detection axis, and
a non-prostrate-state determination mode in which a determination that the livestock animal has been in the non-prostrate state can be made on the basis of the other ones of the output values, the other ones corresponding to the non-prostrate-posture detection, and

after the postural-state determination unit has determined that the livestock animal has been in the prostrate state in the prostrate-state determination mode, the postural-state determination unit is switched from the prostrate-state determination mode into the non-prostrate-state determination mode.
(5) The livestock sensor device according to any one of (1) to (4), in which

the state inference unit
infers that first astasia has developed when the prostrate state has continued for a first duration or longer, and
infers that second astasia that is more urgent than the first astasia has developed when the prostrate state has continued for a second duration that is longer than the first duration, or longer.
(6) The livestock sensor device according to any one of (1) to (5), in which

when the output values include output values that are each larger than a preset output value and that are detected a predetermined number of times or more within a predetermined time period after a determination that the livestock animal has been in the prostrate state has been made, the state inference unit infers that the livestock animal has been in a self-standing-attempt state that accompanies the astasia of the livestock animal.
(7) The livestock sensor device according to any one of (1) to (6), in which

the transmission unit transmits, periodically to the server, a postural data item
that includes a postural-state information item about in which of the prostrate state and the non-prostrate state the livestock animal has been, in which of the prostrate state and the non-prostrate state the livestock animal has been being determined by the postural-state determination unit, and
that does not include the astasia-notification data item.
(8) The livestock sensor device according to (7), in which

the transmission unit transmits the astasia-notification data item with priority over the postural data item.
(9) The livestock sensor device according to (8), in which

the transmission unit is
configured to be capable of executing retry processes in cases where a process of transmitting the postural data item and a process of transmitting the astasia-notification data item to the server fail, and
configured to be capable of executing the retry processes a larger number of times at a time of the process of transmitting the astasia-notification data item than a number of times at a time of the process of transmitting the postural data item.
(10) The livestock sensor device according to any one of (1) to (9), in which

the casing is configured to be capable of being attached to a submental part of the livestock animal.
(11

) A livestock astasia inference method, including

determining in which of a prostrate state and a non-prostrate state a livestock animal has been on a basis of output values from an acceleration sensor; and

inferring whether or not the livestock animal has developed astasia on a basis of a duration of the prostrate state.
(12) The livestock astasia inference method according to (11), further including

inferring that the livestock animal has been in a self-standing-attempt state that accompanies the astasia of the livestock animal when the output values include output values that are each larger than a preset output value and that are detected a predetermined number of times or more within a predetermined time period after a determination that the livestock animal has been in the prostrate state has been made.
(13) A program for causing a computer to carry out a livestock astasia inference method including:

determining in which of a prostrate state and a non-prostrate state a livestock animal has been on a basis of output values from an acceleration sensor; and

inferring whether or not the livestock animal has developed astasia on a basis of a duration of the prostrate state.
(14) A livestock management system, including:

a postural-state determination unit that determines in which of a prostrate posture and a non-prostrate posture a livestock animal has been on a basis of output values from an acceleration sensor;

a state inference unit that infers whether or not the livestock animal has developed astasia on a basis of a duration of the prostrate posture;

a notification-information generation unit that generates an astasia-notification information item including an astasia-notification data item indicating that an inference that the livestock animal has developed the astasia is made when the inference that the livestock animal has developed the astasia is made; and

a notification unit that notifies a user of the astasia-notification information item.
(15) The livestock management system according to (14), in which

the livestock animal include a plurality of livestock animals;

the postural-state determination unit determines in which of the prostrate posture and the non-prostrate posture each of the plurality of livestock animals has been on the basis of the output values from the acceleration sensor;

the livestock management system further includes
a postural-data storage unit that stores a postural data item of each of the plurality of livestock animals, the postural data item
   including a postural-state information item about in which of the prostrate state and the non-prostrate state corresponding one of the plurality of livestock animals has been, and
   not including the astasia-notification data item of the corresponding one of the plurality of livestock animals.
(16) The livestock management system according to (15), in which

the state inference unit infers whether or not each of the plurality of livestock animals has developed astasia on the basis of the duration of the prostrate posture, and

the livestock management system further includes:
a notification-information storage unit that stores the astasia-notification information item of each of the plurality of livestock animals, the astasia-notification information item including the astasia-notification data item indicating that the inference that the corresponding one of the plurality of livestock animals has developed the astasia is made; and
   an analysis unit that analyzes respective risks that the plurality of livestock animals develops the astasia
      on a basis of the astasia-notification information item of each of the plurality of livestock animals, and
      on a basis of the postural data item of each of the plurality of livestock animals.

### REFERENCE SIGNS LIST

1, 1A, 1C, 1D, 1E, 1F sensor device
3, 3A, 3B, 3C, 3E, 3F server
10 casing
100, 100A, 100B, 100C, 100D, 100E, 100F, 100G livestock management system
101 detection unit
102, 102A, 102D, 102E, 102F postural-state determination unit
103, 103A, 103C, 103E state inference unit
104, 104A, 104C, 104F transmission unit
106, 106E notification-information generation unit
107 notification unit
108 postural-data storage unit
109 notification-information storage unit
110 analysis unit

1] A livestock sensor device, comprising:
a postural-state determination unit that determines in which of a prostrate state and a non-prostrate state a livestock animal has been on a basis of output values from an acceleration sensor;
a state inference unit that infers whether or not the livestock animal has developed astasia on a basis of a duration of the prostrate state;
a transmission unit that transmits, to a server, an astasia-notification data item indicating that an inference that the livestock animal has developed the astasia is made when the inference that the livestock animal has developed the astasia is made; and
a casing that is configured to be
capable of housing
the acceleration sensor,
the postural-state determination unit,
the state inference unit, and
the transmission unit, and
capable of being attached to a head of the livestock animal.

2] The livestock sensor device according to claim 1, further comprising
a detection unit that outputs a detection signal when the output values from the acceleration sensor include output values that are each larger than a preset output value and that continue to be detected, wherein the postural-state determination unit determines in which of the prostrate state and the non-prostrate state the livestock animal has been on a basis of the detection signal.

3] The livestock sensor device according to claim 1, wherein
the acceleration sensor has a plurality of detection axes,
the plurality of detection axes includes
a prostration detection axis that enables detection of a highest acceleration in a gravity direction from among the plurality of detection axes in the prostrate state of the livestock animal,
a non-prostration detection axis that enables detection of the highest acceleration in the gravity direction from among the plurality of detection axes in the non-prostrate state of the livestock animal,
the postural-state determination unit
determines that the livestock animal has been in the prostrate state on a basis of ones of the output values, the ones corresponding to the prostration detection axis, and
determines that the livestock animal has been in the non-prostrate state on a basis of other ones of the output values, the other ones corresponding to the non-prostration detection axis.

4] The livestock sensor device according to claim 3, wherein
the postural-state determination unit includes
a prostrate-state determination mode in which a determination that the livestock animal has been in the prostrate state can be made on the basis of the ones of the output values, the ones corresponding to the prostrate-posture detection axis, and
a non-prostrate-state determination mode in which a determination that the livestock animal has been in the non-prostrate state can be made on the basis of the other ones of the output values, the other ones corresponding to the non-prostrate-posture detection, and
after the postural-state determination unit has determined that the livestock animal has been in the prostrate state in the prostrate-state determination mode, the postural-state determination unit is switched from the prostrate-state determination mode into the non-prostrate-state determination mode.

5] The livestock sensor device according to claim 1, wherein
the state inference unit
infers that first astasia has developed when the prostrate state has continued for a first duration or longer, and
infers that second astasia that is more urgent than the first astasia has developed when the prostrate state has continued for a second duration that is longer than the first duration, or longer.

6] The livestock sensor device according to claim 1, wherein
when the output values include output values that are each larger than a preset output value and that are detected a predetermined number of times or more within a predetermined time period after a determination that the livestock animal has been in the prostrate state has been made, the state inference unit infers that the livestock animal has been in a self-standing-attempt state that accompanies the astasia of the livestock animal.

7] The livestock sensor device according to claim 1, wherein
the transmission unit transmits, periodically to the server, a postural data item
that includes a postural-state information item about in which of the prostrate state and the non-prostrate state the livestock animal has been, in which of the prostrate state and the non-prostrate state the livestock animal has been being determined by the postural-state determination unit, and
that does not include the astasia-notification data item.

8] The livestock sensor device according to claim 7, wherein
the transmission unit transmits the astasia-notification data item with priority over the postural data item.

9] The livestock sensor device according to claim 8, wherein
the transmission unit is
configured to be capable of executing retry processes in cases where a process of transmitting the postural data item and a process of transmitting the astasia-notification data item to the server fail, and
configured to be capable of executing the retry processes a larger number of times at a time of the process of transmitting the astasia-notification data item than a number of times at a time of the process of transmitting the postural data item.

10] The livestock sensor device according to claim 1, wherein
the casing is configured to be capable of being attached to a submental part of the livestock animal.

11] A livestock astasia inference method, comprising
determining in which of a prostrate state and a non-prostrate state a livestock animal has been on a basis of output values from an acceleration sensor; and
inferring whether or not the livestock animal has developed astasia on a basis of a duration of the prostrate state.

12] The livestock astasia inference method according to claim 11, further comprising
inferring that the livestock animal has been in a self-standing-attempt state that accompanies the astasia of the livestock animal when the output values include output values that are each larger than a preset output value and that are detected a predetermined number of times or more within a predetermined time period after a determination that the livestock animal has been in the prostrate state has been made.

13] A program for causing a computer to carry out a livestock astasia inference method comprising:
determining in which of a prostrate state and a non-prostrate state a livestock animal has been on a basis of output values from an acceleration sensor; and
inferring whether or not the livestock animal has developed astasia on a basis of a duration of the prostrate state.

14] A livestock management system, comprising:
a postural-state determination unit that determines in which of a prostrate posture and a non-prostrate posture a livestock animal has been on a basis of output values from an acceleration sensor;
a state inference unit that infers whether or not the livestock animal has developed astasia on a basis of a duration of the prostrate posture;
a notification-information generation unit that generates an astasia-notification information item including an astasia-notification data item indicating that an inference that the livestock animal has developed the astasia is made when the inference that the livestock animal has developed the astasia is made; and
a notification unit that notifies a user of the astasia-notification information item.

15] The livestock management system according to claim 14, wherein
the livestock animal includes a plurality of livestock animals;
the postural-state determination unit determines in which of the prostrate posture and the non-prostrate posture each of the plurality of livestock animals has been on the basis of the output values from the acceleration sensor;
the livestock management system further comprises
a postural-data storage unit that stores a postural data item of each of the plurality of livestock animals, the postural data item
including a postural-state information item about in which of the prostrate state and the non-prostrate state corresponding one of the plurality of livestock animals has been, and
not including the astasia-notification data item of the corresponding one of the plurality of livestock animals.

16] The livestock management system according to claim 15, wherein
the state inference unit infers whether or not each of the plurality of livestock animals has developed astasia on the basis of the duration of the prostrate posture, and
the livestock management system further comprises:
a notification-information storage unit that stores the astasia-notification information item of each of the plurality of livestock animals, the astasia-notification information item including the astasia-notification data item indicating that the inference that the corresponding one of the plurality of livestock animals has developed the astasia is made; and
an analysis unit that analyzes respective risks that the plurality of livestock animals develops the astasia
on a basis of the astasia-notification information item of each of the plurality of livestock animals, and
on a basis of the postural data item of each of the plurality of livestock animals.
